SEQUENCE LISTING (59 sequences)

SEQ ID NO: 1 - protein, 27 aa, Homo sapiens
MAALEKDYEEVGVDSVEGEGEEEGEEY

SEQ ID NO: 2 - protein, 27 aa, Homo sapiens
MAALEKDYEEVGVHSVEGEGEEEGEEY

SEQ ID NO: 3 - protein, 26 aa, Homo sapiens
LAALEKDYEEVGVDSVEAEAEEGEEY

SEQ ID NO: 4 - protein, 24 aa, Homo sapiens
MAALEKDYEEVGIDSYEDEDEGEE

SEQ ID NO: 5 - protein, 25 aa, Homo sapiens
MAALEKDYEEVGADSADGEDEGEEY

SEQ ID NO: 6 - protein, 25 aa, Homo sapiens
LAALEKDYEEVGTDSFEEENEGEFF

SEQ ID NO: 7 - protein, 20 aa, Homo sapiens
YQDATAEEEEDFGEEAEEEA

SEQ ID NO: 8 - protein, 21 aa, Homo sapiens
YQDATADEQGEFEEEEGEDEA

SEQ ID NO: 9 - protein, 26 aa, Homo sapiens
YQDATAEEEGEMYEDDEEESEAQGPK

SEQ ID NO: 10 - protein, 20 aa, Homo sapiens
YQDATAEQGEFEEEAEEEVA

SEQ ID NO: 11 - protein, 20 aa, Homo sapiens
YQDATAEEGEFEEEAEEEVA

SEQ ID NO: 12 - protein, 21 aa, Homo sapiens
YQDATAEEEGEFEEEAEEEVA

SEQ ID NO: 13 - protein, 22 aa, Homo sapiens
YQDATANDGEEAFEDEEEEIDG

SEQ ID NO: 14 - protein, 27 aa, Homo sapiens
FDQAKAVLEEDEEVTEEAEMEPEDKGH

SEQ ID NO: 15 - DNA, 21 bp, Artificial
  Forward PCR primer for human class I beta-tubulin isotype (HM40/TUBB)
acctcaggcttctcagttccc

SEQ ID NO: 16 - DNA, 28 bp, Artificial
  Probe for human class I beta-tubulin isotype (HM40/TUBB)
tagccgtcttactcaactgcccctttcc

SEQ ID NO: 17 - DNA, 22 bp, Artificial
  Reverse PCR primer for human class I beta-tubulin isotype (HM40/TUBB)
cagcaaacacaaattctgaggg

SEQ ID NO: 18 - DNA, 22 bp, Artificial
  Forward PCR primer for human class II beta-tubulin isotype (HB9/TUBB2)
gtggaaggaaagaagcatggtc

SEQ ID NO: 19 - DNA, 26 bp, Artificial
  Probe for human class II beta-tubulin isotype (HB9/TUBB2)
actttaggtgtgcgctgggtctctgg

SEQ ID NO: 20 - DNA, 22 bp, Artificial
  Reverse PCR primer for human class II beta-tubulin isotype (HB9/TUBB2)
gtgacaggcaacagtgaagagc

SEQ ID NO: 21 - DNA, 19 bp, Artificial
  Forward PCR primer for human class III beta-tubulin isotype (HB4/TUBB4)
cctcgtcctccccacctag

SEQ ID NO: 22 - DNA, 26 bp, Artificial
  Probe for human class III beta-tubulin isotype (HB4/TUBB4)
ccacgtgtgagctgctcctgtctctg

SEQ ID NO: 23 - DNA, 20 bp, Artificial
  Reverse PCR primer for human class III beta-tubulin isotype (HB4/TUBB4)
aggcctggagctgcaataag

SEQ ID NO: 24 - DNA, 21 bp, Artificial
  Forward PCR primer for human class IVa beta-tubulin isotype (HB5/TUBB5)
tctgacctttgatccgctagg

SEQ ID NO: 25 - DNA, 25 bp, Artificial
  Probe for human class IVa beta-tubulin isotype (HB5/TUBB5)
cccccatctctgaaccctagagccc

SEQ ID NO: 26 - DNA, 19 bp, Artificial
  Reverse PCR primer for human class IVa beta-tubulin isotype (HB5/TUBB5)
tcagccttggagggaaagc

SEQ ID NO: 27 - DNA, 26 bp, Artificial
  Forward PCR primer for human class IVb beta-tubulin isotype (HB2)
ggaagcagtgtgaactctttattcac

SEQ ID NO: 28 - DNA, 21 bp, Artificial
  Probe for human class IVb beta-tubulin isotype (HB2)
cccagcctgtcctgtggcctg

SEQ ID NO: 29 - DNA, 20 bp, Artificial
  Reverse PCR primer for human class IVb beta-tubulin isotype (HB2)
cagcaagtgcacacagtggg

SEQ ID NO: 30 - DNA, 19 bp, Artificial
  Forward PCR primer for human class V beta-tubulin isotype (5-beta/Beta V)
ccctggtgcctcctaccct

SEQ ID NO: 31 - DNA, 24 bp, Artificial
  Probe for human class V beta-tubulin isotype (5-beta/Beta V)
tggccctgaatggtgcactggttt

SEQ ID NO: 32 - DNA, 18 bp, Artificial
  Reverse PCR primer for human class V beta-tubulin isotype (5-beta/Beta V)
gggccgacaccaacacaa

SEQ ID NO: 33 - DNA, 21 bp, Artificial
  Foward PCR primer for human class VI beta-tubulin isotype (HB1-TUBB1)
tgcactcaccattagcttcga

SEQ ID NO: 34 - DNA, 25 bp, Artificial
  Probe for human class VI beta-tubulin isotype (HB1/TUBB1)
acagggactgagggagacaggtggg

SEQ ID NO: 35 - DNA, 20 bp, Artificial
  Reverse PCR primer for human class VI beta-tubulin isotype (HB1/TUBB1)
ccctaatgcctgtcagctgc

SEQ ID NO: 36 - DNA, 17 bp, Artificial
  Forward PCR primer for human MAP4
tgagccggtcaggcaca

SEQ ID NO: 37 - DNA, 24 bp, Artificial
  Probe for human MAP4
accaaccagtccacgctccaaggg

SEQ ID NO: 38 - DNA, 22 bp, Artificial
  Reverse PCR primer for human MAP4
gcatacacacaacaaaatggca

SEQ ID NO: 39 - DNA, 20 bp, Artificial
  Forward PCR primer for human stathmin
cacaaatgaccgcacgttct

SEQ ID NO: 40 - DNA, 20 bp, Artificial
  Probe for human stathmin
tgccccgtttcttgccccag

SEQ ID NO: 41 - DNA, 21 bp, Artificial
  Reverse PCR primer for human stathmin
ggaaggagacaatgcaaacca

SEQ ID NO: 42 - DNA, 19 bp, Artificial
  Forward PCR primer for human class 1 alpha-tubulin isotype (TUBA1/a1)
tgccaacaactatgcccgt

SEQ ID NO: 43 - DNA, 32 bp, Artificial
  Probe for human class 1 alpha-tubuling isotype (TUBA1/k-a1)
tataccattggcaaggagatcattgacccagt

SEQ ID NO: 44 - DNA, 19 bp, Artificial
  Reverse PCR primer for human class 1 alpha-tubulin isotype (TUBA1/k-a1)
tggaacaccaggaagccct

SEQ ID NO: 45 - DNA, 19 bp, Artificial
  Forward PCR primer for human class 3 alpha-tubulin isotype (TUBA2)
tggtgcccaaggatgtgaa

SEQ ID NO: 46 - DNA, 29 bp, Artificial
  Probe for human class 3 alpha-tubulin isotype (TUBA2)
ctattgctgccatcaagaccaagaggacc

SEQ ID NO: 47 - DNA, 20 bp, Artificial
  Reverse PCR primer for human class 3 alpha-tubulin isotype (TUBA2)
gttgatgcccaccttgaagc

SEQ ID NO: 48 - DNA, 20 bp, Artificial
  Forward PCR primer for human class 1 alpha-tubulin isotype (TUBA3/b-a1)
cctcgtgttggaccgaattc

SEQ ID NO: 49 - DNA, 19 bp, Artificial
  Probe for human class 1 alpha-tubulin isotype (TUBA3/b-a1)
tggccgaccagtgcacggg

SEQ ID NO: 50 - DNA, 20 bp, Artificial
  Reverse for human class 1 alpha-tubulin isotype (TUBA3/b-a1)
tggaaaaccaagaagccctg

SEQ ID NO: 51 - DNA, 21 bp, Artificial
  Forward PCR primer for human class 4 alpha-tubulin isotype (TUBA4)
ggaaggagttcatcgacctgc

SEQ ID NO: 52 - DNA, 23 bp, Artificial
  Probe for human class 4 alpha-tubulin isotype (TUBA4)
accggattcggaagctggctgac

SEQ ID NO: 53 - DNA, 19 bp, Artificial
  Reverse PCR primer for human class 4 alpha-tubulin isotype (TUBA4)
tggaacaccaggaagccct

SEQ ID NO: 54 - DNA, 19 bp, Artificial
  Forward PCR primer for human class 6 alpha-tubulin isotype (TUBA6)
cccgagggcactacaccat

SEQ ID NO: 55 - DNA, 24 bp, Artificial
  Probe for human class 6 alpha-tubulin isotype (TUBA6)
cagtgcaccggtcttcagggcttc

SEQ ID NO: 56 - DNA, 20 bp, Artificial
  Reverse PCR primer for human class 6 alpha-tubulin isotype (TUBA6)
aaccagttcccccaccaaag

SEQ ID NO: 57 - DNA, 19 bp, Artificial
  Forward PCR primer for human class 8 alpha-tubulin isotype (TUBA8)
tggtgcccaaggatgtgaa

SEQ ID NO: 58 - DNA, 26 bp, Artificial
  Probe for human class 8 alpha-tubulin isotype (TUBA8)
ttgctgccatcaagaccaagaggacc

SEQ ID NO: 59 - DNA, 20 bp, Artificial
  Reverse PCR primer for human class 8 alpha-tubulin isotype (TUBA8)
gttgatgcccaccttgaagc

(19) United States
(12) Patent Application Publication
(10) Pub. No.: US 2010/0190843 A1
(43) Pub. Date: Jul. 29, 2010

(54) Tubulin Isotype Screening in Cancer Therapy Using Halichondrin B Analogs

(75) Inventors: Sergei Agoulnik, Andover, MA (US); Galina Kuznetsov, Lexington, MA (US); Bruce A. Littlefield, Andover, MA (US)

Correspondence Address: Clark & Elbing LLP / Eisai, 101 Federal Street, Suite 1500, Boston, MA, 02110, US

(73) Assignee: Eisai R&D Management Co., Ltd., Tokyo (JP)

(21) Appl. No.: 12/534,277

(22) Filed: Aug. 3, 2009

Related U.S. Application Data
(63) Continuation of application No. 11/299,260, filed on Dec. 7, 2005, now abandoned.
(60) Provisional application No. 60/634,734, filed on Dec. 9, 2004.

(51) Int. Cl. A61K 31/335; C07H 21/04; G01N 33/68; C12Q 1/02; G01N 33/48; C12Q 1/68; C40B 30/04; G01N 33/53; A61P 35/00

(52) U.S. Cl. 514/450; 536/23.1; 436/86; 435/29; 436/94; 435/6; 506/9; 436/501

(57) ABSTRACT

Chemotherapeutic agents that interfere with microtubule assembly or disassembly in the cell are potent inhibitors of cell replication. Examples of such agents include halichondrin B analogs. It has been shown that the susceptibility of certain cancers to analogs of halichondrin B correlates with the expression of particular tubulin isotypes or other microtubule-associated proteins such as MAP-4 and stathmin. Correlations such as these may be used in identifying patients suitable for treatment using a particular chemotherapeutic agent. Such a system avoids treating patients with cytotoxic compounds where there is a minimal or no effect on the cancer. The invention also provides a system of establishing these correlations for different compounds and cancer types. The system will be particularly useful in establishing correlations between anti-microtubule agents and cancers such as lung, breast, and ovarian cancer. Kits and reagents useful in practicing the invention are also provided.

### RELATED APPLICATIONS

The present application is a continuation of, and claims priority from, U.S. Ser. No. 11/299,260, filed Dec. 7, 2005, which claims benefit under 35 U.S.C. §119(e) of U.S. Ser. No. 60/634,734, filed Dec. 9, 2004, incorporated herein by reference.

### BACKGROUND OF THE INVENTION

In many instances, the treatment of cancer involves the systemic administration of cytotoxic compounds to the patient suffering with the disease. Since cancer cells are dividing more quickly than normal cells in the patient, these cytotoxic compounds exert a greater effect on the cancer cells than on the patient's normal cells. However, this phenomenon does not prevent these compounds from having severe, adverse side effects. These side effects may range from weight loss, diarrhea, nausea, and hair loss to more severe side effects such as anemia, secondary cancers, organ toxicity, and even death. Unfortunately, a significant number of patients do not respond or do not receive substantial benefit from treatment; however, they do suffer the side effects. Therefore, it would be very useful to be able to predict which patients will respond to treatment before the first dose is administered. However, in many cases it is difficult to determine whether a cancer will respond to treatment without actually administering the drug to the patient. And in many cases the treatment may be continued for several weeks before it is clear that the cancer is resistant or not susceptible to the treatment. Various systems have been designed to classify tumors (e.g., pathological classification, tumor markers) and thereby predict drug efficacy. However, there remains a need to better predict whether a cancer will respond to a particular chemotherapeutic agent.

With the advent of genomics and proteomics, it is generally hoped that characterizing the expression of genes in the cancer cell will allow the clinician to custom tailor the cancer therapy for the patient to provide an optimized therapy with the greatest effect on the cancer and the least number of side effects. In certain instances, the expression of a gene may indicate that the cancer will not respond to a particular drug or class of drug. In other cases, the expression of a gene may indicate that the cancer will respond. Being able to predict whether a patient's cancer will respond to treatment allows a physician to tailor the treatment to maximize the likelihood of successful treatment of the cancer while minimizing the risk of adverse side effects.

It has been shown that compounds which interfere with microtubule polymerization are not effective in treating certain cancers. For example, paclitaxel (Taxol) has been found to not be effective in treating cells expressing class II β-tubulin (Haber et al. J. Biol. Chem. 270(52):31269-31275, 1995; incorporated herein by reference). There remains a need for a system of selecting patients whose cancers are susceptible to agents known to interfere with microtubule assembly. Such a diagnostic system would allow only the patients whose cancers are susceptible to the agent to be treated with these cytotoxic agents.

### SUMMARY OF THE INVENTION

The present invention provides a system, including, for example, methods, apparatus, materials, polynucleotides, reagents, software, kits, etc. for predicting whether a cancer patient will respond to treatment with a particular chemical compound. By assessing the expression of tubulin isotypes or other microtubule-associated biomolecules in the cancer cells of the patient, one may evaluate whether the cancer will respond to a particular chemical compound. In this manner, the invention may be used to select and/or treat a patient with cancer (e.g., breast cancer, lung cancer, ovarian cancer, prostate cancer, pancreatic cancer, etc.). The inventive method is particularly useful in predicting whether the patient will respond to an organic compound that interferes with microtubule assembly or disassembly, binds microtubules, or binds tubulin.

In certain embodiments the compounds tested for efficacy are halichondrin B analogs having anti-cancer and/or anti-mitotic activity. In general, the analogs have the formula (I):

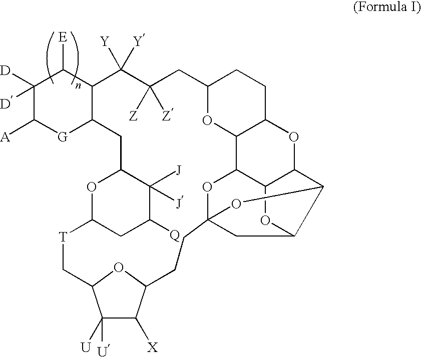

wherein A is a C₁₋₆ saturated or C₂₋₆ unsaturated hydrocarbon skeleton, the skeleton being unsubstituted or having between 1 and 13 substituents, preferably between 1 and 10 substituents, e.g., at least one substituent selected from cyano, halo, azido, Q₁, and oxo, wherein each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁ and O(CO)NR₂R₁, and the number of substituents can be, for example, between 1 and 6, 1 and 8, 2 and 5, or 1 and 4;

wherein each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl (e.g., p-fluorophenyl or p-chlorophenyl), C₆₋₁₀ hydroxyaryl, C₁₋₄ alkoxy-C₆ aryl (e.g., p-methoxyphenyl, 3,4,5-trimethoxyphenyl, p-ethoxyphenyl, or 3,5-diethoxyphenyl), C₆₋₁₀ aryl-C₁₋₆ alkyl (e.g., benzyl or phenethyl), C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl;

wherein each of D and D′ is independently selected from R₃ and OR₃, wherein R₃ is H, C₁₋₃ alkyl, or C₁₋₃ haloalkyl;

wherein the value for n is 1 or preferably 0, thereby forming either a six-membered or five-membered ring, wherein the ring can be unsubstituted or substituted, where E is —R₅ or —OR₅, and can be a heterocyclic radical or a cycloalkyl, e.g., where G is S, SH₂, NR₆, or preferably O;

wherein each of J and J′ is independently H, C₁₋₆ alkoxy, or C₁₋₆ alkyl; or J and J′ taken together are ═CH₂ or —O-(straight or branched C₁₋₅alkylene or alkylidene)-O—, such as exocyclic methylidene, isopropylidene, methylene, or ethylene;

wherein Q is C₁₋₃ alkyl, and is preferably methyl;

wherein T is methylene, ethylene, or ethenylene, optionally substituted with (CO)OR₇, where R₇ is H or C₁₋₆ alkyl;

wherein each of U and U′ is independently H, C₁₋₆ alkoxy, or C₁₋₆ alkyl; or U and U′ taken together are ═CH₂ or —O-(straight or branched C₁₋₅alkylene or alkylidene)-O—;

wherein X is H or C₁₋₆ alkoxy;

wherein each of Y and Y′ is independently H or C₁₋₆ alkoxy; or Y and Y′ taken together are ═O, ═CH₂, or —O-(straight or branched C₁₋₅alkylene or alkylidene)-O—;

wherein each of Z and Z′ is independently H or C₁₋₆ alkoxy; or Z and Z′ taken together are ═O, ═CH₂, or —O-(straight or branched C₁₋₅alkylene or alkylidene)-O—. Halichondrin B analogs, the synthesis, methods of treatment, and pharmaceutical compositions thereof are described in U.S. Pat. Nos. 6,214,865; 6,365,759; 6,469,182; and 6,653,341; each of which is incorporated herein by reference; and U.S. patent applications, U.S. Ser. No. 60/576,642, filed Jun. 3, 2004; U.S. Ser. No. 60/626,769, filed Nov. 10, 2004; and U.S. Ser. No. 10/687,526, filed Oct. 16, 2003; each of which is incorporated herein by reference. One particularly useful analog of halichondrin B is E7389 which has the formula (VI):

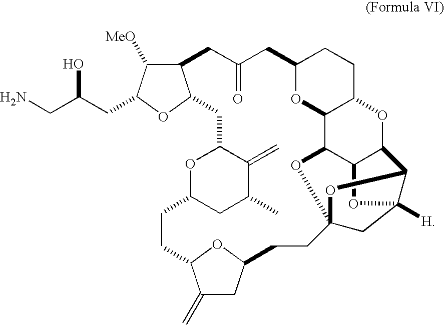

In one aspect, the inventive system includes a method of identifying patients for treatment by predicting whether a patient's cancer is susceptible to treatment with a particular chemical compound based on the expression levels or protein levels of tubulin isotypes and/or microtubule-associated proteins by the cancer cells. In certain embodiments, the cancer of the patient expresses a particularly relevant tubulin isotype or other microtubule-associated biomolecule two times, three times, four times, or five times higher relative to a control cell or population of cells. The method allows for the classification of patients as good or bad candidates for treatment with a particular compound. If the patient is identified as a “good” candidate for treatment, the patient may optionally be administered a therapeutically effective amount of the compound.

In the method, a sample from the cancer is obtained, and the expression levels or protein levels of one or more tubulin isotypes or tubulin-associated biomolecules is determined. Based on the detected expression levels or protein levels, one can predict based on the correlations described herein whether the chemical compound such as a halichondrin B analog will be effective in treating the patient with the cancer. The method is particularly useful in predicting the efficacy in treating cancers susceptible to anti-microtubule agents or microtubule binding agents, e.g., breast cancer, ovarian cancer, and lung cancer. Any available technique for detecting the expression of a gene or detecting protein levels may be used. For example, expression levels or protein levels of tubulin isotypes or tubulin-associated biomolecules may be detected by PCR, gene chips, immunoassays, or mass spectroscopy.

In certain embodiments, the diagnostic method of identifying a patient with cancer for treatment with a halichondrin B analog (as described above in Formula I) includes the steps of:

(a) obtaining a sample from the cancer of a patient; and

(b) analyzing the sample for expression levels or protein levels of at least one marker selected from the group consisting of α-tubulin isotypes, β-tubulin isotypes, and microtubule-associated biomolecules, wherein a correlation exists between sensitivity to a chemical compound and expression levels or protein levels of the marker; and

(c) identifying the patient based on expression levels or protein levels of the marker.

The present inventors have demonstrated that the expression of the class III isotype of β-tubulin in breast cancer cells correlates with sensitivity to certain tubulin-binding agents, including particular analogs of halichondrin B (e.g., E7389) and hemiasterlin (e.g., E7974) (see Example 1). The inventors have further demonstrated that expression of the class III isotype of β-tubulin expression correlates with sensitivity to certain tubulin-binding agents, including particular hemiasterlin analogs (see Example 1 and U.S. patent application U.S. Ser. No. 60/634,756, filed Dec. 9, 2004, entitled “Tubulin Isotype Screening in Cancer Therapy using Hemiasterlin Analogs”, which is incorporated herein by reference). Other β-tubulin isotypes have also been found to correlate with sensitivity to E7389 including class IVb, class V, and class VI β-tubulin isotypes, and in particular, class III, class IVb, and class V β-tubulin isotypes. Class 1 α-tubulin isotype (TUBA1/k-α1) has also been found to correlate with sensitivity to E7389.

In another aspect, the present invention provides a method for treating patients identified as being “good” candidates for treatment with halichondrin B analogs. In certain embodiments, the method of selecting a compound for treating a patient with cancer based on the expression level or protein level of at least one marker selected from the group consisting of α-tubulin isotypes, β-tubulin isotypes, and microtubule-associated biomolecules comprising administering to the patient a compound of the formula (I) as described above, based on the expression level or protein level of at least one marker selected from the group consisting of α-tubulin isotypes, β-tubulin isotypes, and microtubule-associated biomolecules.

The inventive system also provides a system for determining the correlation of tubulin isotype or microtubule-associated biomolecule expression levels or protein levels with sensitivity of a cancer to other cytotoxic agents. In this method, various cancer cells lines are exposed to the test compound. The cell growth inhibition is tested, and the expression levels or protein levels of particular tubulin isotypes and microtubule-associated biomolecules is assessed. The correlations between sensitivity of cell lines to the test agent and expression level of genes of interest are then calculated. A conventional threshold of correlation coefficient (Pearson r) is considered significant with a p-value of 0.05 or less. A p-value of 0.20 or less, 0.15 or less, or 0.10 or less may also be used. These correlations may then be used in the inventive system for selecting and treating patients using the test compound.

In a certain embodiments, the method of determining a correlation between susceptibility to a chemical compound and expression of a marker gene (e.g., tubulin isotype, microtubule-associated biomolecule (e.g., MAP4, stathmin, Tau)) includes:

(a) providing a cell, typically a cancer cell;

(b) contacting the cell with a compound of the formula (I) as described above;

(c) assaying the cell for growth inhibition;

(d) determining the expression of tubulin isotypes or microtubule-associated genes in the cell; and

(e) determining a correlation between expression levels or protein levels of one or more tubulin isotypes or microtubule-associated biomolecules and susceptibility to the compound tested. In certain embodiments, the correlation is determined by using linear regression analysis. In other embodiments, the correlation is determined using multiple stepwise regression analysis.

In another aspect, the invention provides a screening method for identifying compounds that are useful for treating cancer cells expressing a particular tubulin isotype or tubulin-associated protein. The test compounds are contacted with cells (e.g., cancer cell lines) for a particular length of time. The inhibition of growth of the cells is determined, and the expression levels or protein levels of tubulin isotypes and microtubule-associated biomolecules is assessed. These data may then be used to establish a correlation between the sensitivity of a cell expressing a particular tubulin isotype or microtubule-associated biomolecule to the test compound. A conventional threshold of correlation coefficient (Pearson r) is considered significant with a p-value of 0.05 or less. A p-value of 0.20 or less, 0.15 or less, or 0.10 or less may be used. This method may be used to identify clinical candidates or to identify lead compounds in the search for a clinical candidate. Such a system for screening compounds is particularly useful in the search for a chemical compound to treat cancers that are resistant to other known chemotherapeutic agents. For example, it has been shown that paclitaxel (Taxol®) is not effective in treating cells expressing class II β-tubulin (Haber et al. J. Biol. Chem. 270(52):31269-31275, 1995; incorporated herein by reference). The inventive screening method would be useful in the search for compounds that would be effective in cancers expressing class II β-tubulin or any other tubulin isotype or microtubule-associated biomolecule.

The invention also includes kits useful in the practice of the screening, classification, or identification methods described above. The kits may contain reagents such as enzymes, buffers, nucleotides, polynucleotides such as primers and probes, test compounds (e.g., anti-neoplastic agents), cell lines, etc. for practicing the method. Where the method uses PCR, reagents for PCR such as primers specific for tubulin isotypes or microtubule-associated proteins, polymerases, nucleotides, control templates, buffers, etc. may be included in the kits. Where the method uses an immunoassay to detect gene expression, reagents such as antibodies directed to one or more tubulin isotypes or tubulin- or microtubule-associated proteins or other biomolecules may be included in the kits. Gene chips with nucleotide sequences complementary to regions of tubulin isotypes or microtubule-associated genes may also be provided in kits for assessing gene expression. The kits may also contain tools and reagents for obtaining a sample of the cancer, e.g., syringes, needles, storage containers, buffers, etc. The kits may contain materials for extracting RNA from the cancer cells such as poly-TTTTT resins.

The present invention also provides polynucleotides useful as probes or primers, for example to detect the expression levels or protein levels of one or more tubulin isotypes or microtubule-associated biomolecules. Particularly useful probes or primers bind to the mRNA or cDNA of an isotype of tubulin specifically without cross-reacting with other isotypes (e.g., the probes or primers may take advantage of the sequence variations among isotypes seen at the C-termini of tubulins). Primers and probes may also be directed to the mRNAs or cDNAs of other microtubule-associated biomolecules. In certain embodiments, the PCR primers and the probes are useful in determining the expression levels of tubulin isotypes, particularly α-tubulin isotypes and β-tubulin isotypes. In other embodiments, the PCR primers and the probes are useful in determining the expression levels of microtubule-associated biomolecules such as Tau, stathmin, and MAP4.

### DEFINITIONS

The following are chemical terms used in the specification and claims:

The term acyl as used herein refers to a group having the general formula —C(═O)R, where R is alkyl, alkenyl, alkynyl, aryl, carbocylic, heterocyclic, or aromatic heterocyclic. An example of an acyl group is acetyl.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-10 carbon atoms. In another embodiment, the alkyl group employed contains 1-8 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents.

The term alkoxy as used herein refers to a saturated (i.e., alkyl-O—) or unsaturated (i.e., alkenyl-O— and alkynyl-O—) group attached to the parent molecular moiety through an oxygen atom. In certain embodiments, the alkyl group contains 1-20 aliphatic carbon atoms. In certain other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples include, but are not limited to, methoxy, ethoxy, propoxy, isopropoxy, n-butoxy, tert-butoxy, i-butoxy, sec-butoxy, neopentoxy, n-hexoxy, and the like.

The term alkenyl denotes a monovalent group derived from a hydrocarbon moiety having at least one carbon-carbon double bond by the removal of a single hydrogen atom. In certain embodiments, the alkenyl group employed in the invention contains 1-20 carbon atoms. In some embodiments, the alkenyl group employed in the invention contains 1-10 carbon atoms. In another embodiment, the alkenyl group employed contains 1-8 carbon atoms. In still other embodiments, the alkenyl group contains 1-6 carbon atoms. In yet another embodiments, the alkenyl group contains 1-4 carbons. Alkenyl groups include, for example, ethenyl, propenyl, butenyl, 1-methyl-2-buten-1-yl, and the like.

The term alkynyl as used herein refers to a monovalent group derived form a hydrocarbon having at least one carbon-carbon triple bond by the removal of a single hydrogen atom. In certain embodiments, the alkynyl group employed in the invention contains 1-20 carbon atoms. In some embodiments, the alkynyl group employed in the invention contains 1-10 carbon atoms. In another embodiment, the alkynyl group employed contains 1-8 carbon atoms. In still other embodiments, the alkynyl group contains 1-6 carbon atoms. Representative alkynyl groups include, but are not limited to, ethynyl, 2-propynyl (propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. In certain embodiments, the alkyl group contain 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contain 1-6 aliphatic carbon atoms. In yet other embodiments, the alkyl group contain 1-4 aliphatic carbon atoms. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of thioalkoxyl moieties include, but are not limited to, methylthio, ethylthio, propylthio, isopropylthio, n-butylthio, and the like.

The term aryl as used herein refers to an unsaturated cyclic moiety comprising at least one aromatic ring. Aryl groups may contain 5 to 15 carbon atoms, preferably from 5 to 12, and may include 5- to 7-membered rings. In certain embodiments, aryl group refers to a mono- or bicyclic carbocyclic ring system having one or two aromatic rings including, but not limited to, phenyl, naphthyl, tetrahydronaphthyl, indanyl, indenyl, and the like. Aryl groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide. In addition, substituted aryl groups include tetrafluorophenyl and pentafluorophenyl.

The term carboxylic acid as used herein refers to a group of formula —CO₂H.

The terms halo and halogen as used herein refer to an atom selected from fluorine, chlorine, bromine, and iodine.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl) amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl, as used herein, refers to an amide group of the formula —CONH₂.

The term carbonyldioxyl, as used herein, refers to a carbonate group of the formula —O—CO—OR.

The term hydrocarbon, as used herein, refers to any chemical group comprising hydrogen and carbon. The hydrocarbon may be substituted or unsubstitued. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term thiohydroxyl or thiol, as used herein, refers to a group of the formula —SH.

The term ureido, as used herein, refers to a urea group of the formula —NH—CO—NH₂.

The following are more general terms used throughout the present application:

“Antibody”: The term antibody refers to an immunoglobulin or fragment of an immunoglobulin, whether natural or wholly or partially synthetically produced. All derivatives thereof which maintain specific binding ability are also included in the term. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in the present invention. In certain embodiments, the antibodies useful in the present invention are specific for a particular maker. The antibody is preferably specific for a particular isotype of tubulin without cross-reacting with another tubulin isotype. In certain embodiments, the antibody is labeled (e.g., radioactive isotope, fluorescent dye), tagged (e.g., alkaline phosphatase), or derivatized to make it detectable.

“Antibody fragment”: The term antibody fragment refers to any derivative of an antibody which is less than full-length. Preferably, the antibody fragment retains at least a significant portion of the full-length antibody's specific binding ability. Examples of antibody fragments include, but are not limited to, Fab, Fab', F(ab')₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a domesticated animal. An animal may be a transgenic animal. In certain preferred embodiments, the animal is a human.

“Associated with”: When two entities are “associated with” one another as described herein, they are linked by a direct or indirect covalent or non-covalent interaction. Preferably, the association is covalent (e.g., amide, disulfide, or ester linkage). Desirable non-covalent interactions include hydrogen bonding, van der Waals interactions, hydrophobic interactions, magnetic interactions, electrostatic interactions, etc.

“Chemical compound”: In general, the term “chemical compound” as used herein refers to any agent that can be used a chemotherapeutic agents to inhibit the growth of or kill cells or is being tested for its ability to inhibit the growth of or kill cells. Specifically, agents that kill or inhibit the growth of cancer cells are included. The chemical compound may be an organic or inorganic compound. Preferred chemical compounds are organic compounds, particularly small molecules. In certain embodiments, the chemical compound is a halichondrin B analog as described herein. Chemical compound may also refer to biomolecules such as proteins, peptides, oligonucleotides, polynucleotides, fats, lipids, etc.

“Effective amount”: In general, the “effective amount” of a chemical compound refers to the amount necessary or sufficient to elicit the desired biological response. As will be appreciated by those of ordinary skill in this art, the effective amount of a chemical compound may vary depending on such factors as the desired biological endpoint, the compound to be delivered, the disease being treated, the target tissue, etc. In certain embodiments, the effective amount of the compound is the amount necessary to achieve remission or a cure.

“Homologous” or “homologue”: The term “homologous”, as used herein is an art-understood term that refers to nucleic acids or polypeptides that are highly related at the level of nucleotide or amino acid sequence. Nucleic acids or polypeptides that are homologous to each other are termed “homologues.”

The term “homologous” necessarily refers to a comparison between two sequences. In accordance with the invention, two nucleotide sequences are considered to be homologous if the polypeptides they encode are at least about 50-60% identical, preferably about 70% identical, for at least one stretch of at least 20 amino acids. Preferably, homologous nucleotide sequences are also characterized by the ability to encode a stretch of at least 4-5 uniquely specified amino acids. Both the identity and the approximate spacing of these amino acids relative to one another must be considered for nucleotide sequences to be considered homologous. For nucleotide sequences less than 60 nucleotides in length, homology is determined by the ability to encode a stretch of at least 4-5 uniquely specified amino acids.

“Isolated”: The term “isolated”, as used herein, refers to a chemical or biological entity that 1) does not exist in nature; 2) is produced or purified through a process that requires the hand of man; 3) is separated from at least some of the components with which it is associated in nature; and/or 4) is separated from at least some of the components with which is associated when originally produced.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2″-deoxyribose, arabinose, and hexose), and/or modified phosphate groups (e.g., phosphorothioates and 5″-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Microtubule-associated biomolecules”: As used herein, the term microtubule-associated proteins is meant to include any protein, polynucleotide, or other biomolecule found to be directly or indirectly involved in the assembly or disassembly of microtubules in the cells. Examples include various isotypes of tubulin (polymerized and unpolymerized), biomolecules that are associated with the tubulin monomers, biomolecules that are associated microtubules (e.g., microtubule-associated proteins (Type I and II) such as MAP4, MAP2c, Tau, and XMAP215; CLIP-170; EBI; p150), enzymes that degrade tubulin, biomolecules that increase or decrease the transcription, translation, or levels of tubulin, centrioles, centrosomes, bacterial protein FtsZ, microtubule organizing center (MTOC), protein phosphatases such as phosphatases that dephosphorylate MAPs, biomolecules in growth factor signal cascades, protein kinases such as kinases that catalyze the phosphorylation of MAPs, XMAP215, and catastrophe-promoting proteins (catastrophins) such as stathmin and XKCM1.

“Multi-drug resistant”: The term “multi-drug resistant” as applied to a cancer or cancer cell line refers to the simultaneous resistance to a variety of chemically unrelated chemotherapeutic agents. Multi-drug resistance is a major cause of cancer treatment failure. The multi-drug resistant phenotype is typically associated with the expression of P-glycoprotein (Pgp) or multi-drug resistance protein (MRP), two transmembrane transporter protein capable of pumping toxic agents out of cancer cells. Multi-drug resistance may be present initially in a cancer or it may develop over time. The expression of Pgp or MRP in a cell may lead to the concentration of a chemotherapeutic agent being reduced by 50-fold to 1000-fold, rendering the agent useful in treating the cancer.

“Paclitaxel resistant”: The term “paclitaxel resistant” as applied to a cancer or cancer cell line refers to resistance to paclitaxel or other taxane chemotherapeutic agent. In certain embodiments, a patient's cancer may be classified as paclitaxel-resistant after the patient has received chemotherapy treatment with paclitaxel or another taxane chemotherapeutic agent and the cancer failed to respond (e.g., no decrease in tumor burden, no inhibition of growth, etc.). In other embodiments, a cancer may be paclitaxel resistant if it does not respond to paclitaxel at a concentration of 0.001 μM, 0.1 μM, 1 μM, 2 μM, or 5 μM. In certain embodiments, the paclitaxel resistant cancer or cell line is 100-fold, 1000-fold, or 1500-fold less susceptible to paclitaxel. In certain embodiments, the paclitaxel-resistant cancer or cell line expresses P-glycoprotein (Pgp) or multi-drug resistance protein (MRP).

“Tubulin isotypes”: Tubulin, the building blocks of microtubules, comes in three forms α-tubulin, β-tubulin, and γ-tubulin. In humans, there exist six isotypes of α-tubulin and seven isotypes of β-tubulin. The isotypes of α-tubulin are TUBA1 (NCBI protein database accession number 177403), TUBA2 (NCBI protein database accession number. CAA25855), TUBA3 (NCBI protein database accession number Q13748), TUBA4 (NCBI protein database accession number A25873), TUBA6 (NCBI protein database accession number Q9BQE3), and TUBA8 (NCBI protein database accession number Q9NY65). The seven isotypes of β-tubulin are class I isotype, gene HM40/TUBB (NCBI protein database accession number AAD33873); class II isotype, gene Hb9/TUBB2 (NCBI protein database accession number AAH01352); class III isotype, gene Hb4/TUBB4 (NCBI protein database accession number AAH00748); class IVa isotype, gene Hb5/TUBB5 (NCBI protein database accession number P04350, NP_{—}006078); class IVb isotype, gene Hb2 (NCBI protein database accession number P05217); class V isotype, gene 5-beta/BetaV (NCBI protein database accession number NP_{—}115914); and class VI isotype, gene Hb1/TUBB1 (NCBI protein database accession number NP_{—}110400). The entries including the sequences of the above tubulin isotypes from the NCBI protein database are incorporated herein by reference. As would be appreciated by those of skill in this art, other species may have different isotypes of tubulin.

### BRIEF DESCRIPTION OF THE DRAWING

FIG. 1 shows beta-tubulin isotype expression in breast cancer cell lines. Tubulin gene expression was normalized to GAPDH mRNA and plotted as ΔC_{T}.

FIG. 2 shows linear correlations between sensitivity to E7389 and expression level of beta-tubulin isotype genes.

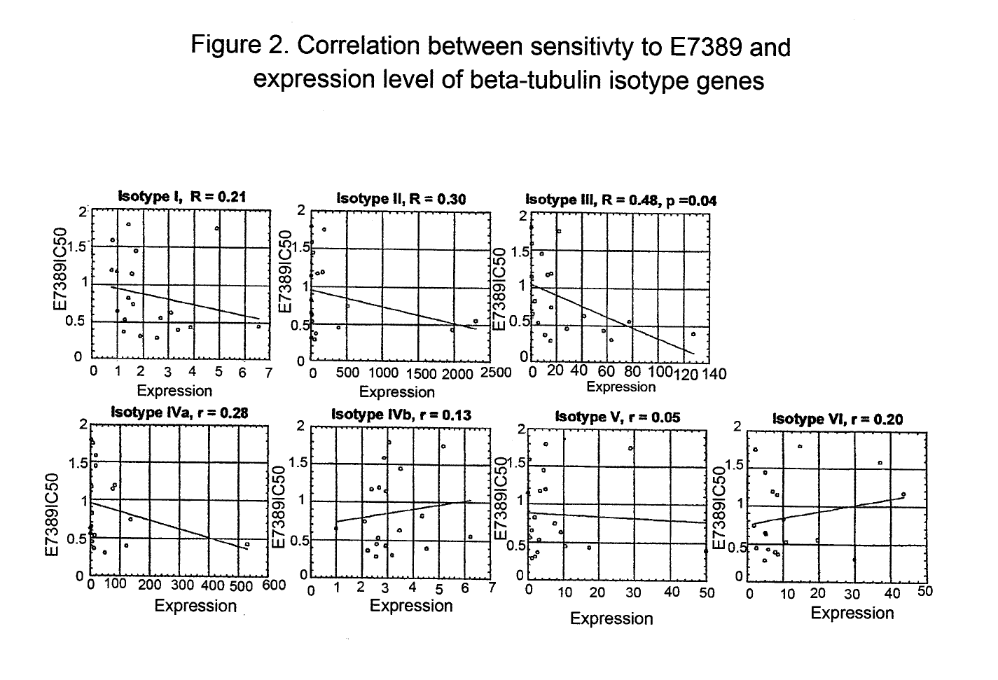

FIG. 3 shows linear correlations between sensitivity to E7974 and expression level of beta-tubulin isotype genes.

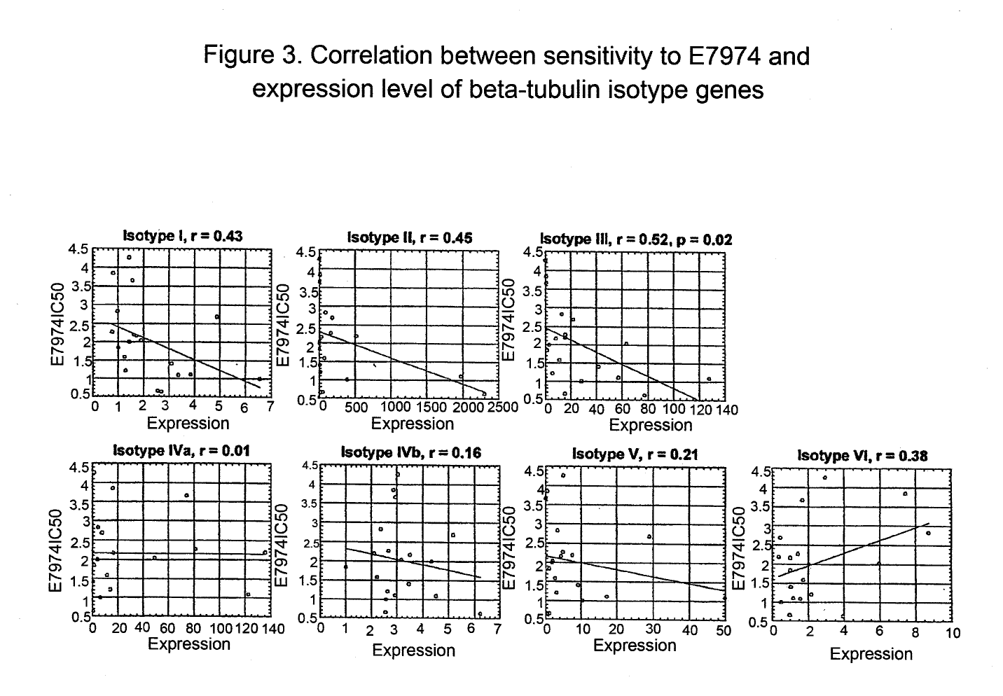

### DETAILED DESCRIPTION OF CERTAIN PREFERRED EMBODIMENTS OF THE INVENTION

The present invention provides methods and materials for identifying patients with cancer that are candidates for treatment with a particular chemotherapeutic agent, or conversely, that would not be candidates for treatment with a particular chemotherapeutic agent. Specifically, the patient with cancer is selected for treatment if the cancer is susceptible to the chemotherapeutic agent, and the patient is not selected if the agent would not affect the cancer. The invention additionally provides methods and materials for treating a patient if the patient has been selected for treatment. Finally, the invention provides methods and materials for identifying chemical compounds that affect microtubule assembly/disassembly in cancer cells expressing tubulin isotypes or tubulin-associated proteins. Using cancer cell lines, correlations between the expression of certain genes and the test compound are determined using statistical methods known in the art.

### Selection of Cancer Patients for Treatment

In selecting a patient for a chemotherapeutic regimen, the cancer of the patient is susceptible to the chemotherapeutic agent to be delivered. For examples, the agent may cure the patient, reduce tumor burden, prevent metathesis, or prevent further growth of the cancer. The side effects of the agent, the condition of the patient, the prognosis, the staging of the cancer, the success or lack of success using other treatment options, etc. may also be considered in making the decision of whether to select the patient for treatment. These additional factors for consideration are apparent to a treating physician.

The inventive system for selecting a patient may be used in selecting any animal for treatment. In certain embodiments, the animal is a mammal; however, birds, reptiles, fish, or other animals may also be selected using the inventive system. In certain embodiments, the patient is a human. In other embodiments, the patient is a domesticated animal (e.g., dog, cat, sheep, goat, pigs, cow, horse, etc.). In yet other embodiments, the patient is an experimental animal (e.g., mice, rats, other rodents, dog, pig, monkeys, other primates, etc.). In sum, any animal species may be selected or not selected for treatment using the inventive system.

The patient typically has cancer; however, the patient may have any abnormal growth of cells, whether it is cancerous or benign, to be screened using the inventive system. Cancers include cancers of any origin (e.g., skin, lung, breast, epithelial cells, mesenchymal cells, mesoderm derived cells, etc.), severity (e.g., poor or favorable prognosis, metastasis or not), pathology (e.g., degree of dysplasia, anaplastic, lack of differentiation), or location (e.g., vital organ, primary tumor or metastasis). In certain embodiments, the cancer is skin cancer (e.g., melanoma), brain cancer (e.g., glioblastoma), lung cancer, stomach cancer, liver cancer, pancreatic cancer, colon cancer, breast cancer, ovarian cancer, testicular cancer, prostate cancer, bladder cancer, kidney cancer, cancer of an endocrine gland, bone cancer, leukemia, sarcoma, lymphoma, or muscle cancer. In certain embodiments, the patient suffers from breast cancer, ovarian cancer, lung cancer, pancreatic cancer (e.g., pancreatic adenocarcinoma), or prostate cancer. In other embodiments, the patient has been diagnosed with breast cancer, ovarian cancer, or lung cancer. In yet other embodiments the patient has breast cancer.

Typically, the cancer of the patient has shown susceptibility to the chemical compound being considered. The cancer may have shown susceptibility to the compound in in vitro or in vivo testing. For example, the cancer may have been responsive to the compound in other patients or in animal models of the cancer. The growth of cancer cell lines may be inhibited by the administration of the compound being considered, or the compound may be cytotoxic to the cell line.

As described below, one aspect of this invention identifies cancers susceptible to microtubule-stabilizing or -destabilizing agents (i.e., anti-microtubule agents) such halichondrin B analogs, hemiasterlin analogs, paclitaxel (Taxol), taxotere, Vinca alkaloids (e.g., vinblastine), colchicine, etc. These cancers include breast cancer, ovarian cancer, and lung cancer. In certain embodiments, prostate cancer may be included. In certain embodiments, the cancer has been shown to be susceptible to halichondrin B analogs, particularly E7389.

The method of identifying a patient for treatment with a chemical compound includes obtaining a sample from the cancer of the patient and determining whether a particular tubulin isotype or microtubule-associated biomolecule is present at particular levels (or within a range of levels) in the cancer cells. In certain embodiments, the presence of certain levels of a tubulin isotype or microtubule-associated biomolecule correlates with susceptibility, or lack thereof, to a particular compound. In certain embodiments, the cancer cells express the marker at a level at least approximately 50% higher than that observed in a control cell or population of cells. In certain embodiments, the cancer cells express the marker at least two times the level observed in a control cell or population of cells. In certain embodiments, the cancer cells express the marker at least three times the level observed in a control cell or population of cells. In certain embodiments, the cancer cells express the marker at least four times the level observed in a control cell or population of cells. In certain embodiments, the cancer cells express the marker at least five times the level observed in a control cell or population of cells. In other embodiments, the cancer cells express the marker at a level at least approximately 75% lower than that observed in a control cell or population of cells. In yet other embodiments, the cancer cells express the marker at a level at least approximately 50% lower than that observed in a control cell or population of cells. In certain embodiments, the cancer cells express the marker at a level at least approximately 25% lower than that observed in a control cell or population of cells. In still other embodiments, the cancer cells do not express the marker.

The patient may be identified as a “good” candidate for treatment with the compound, or the patient may be identified as a “bad” candidate for treatment with the compound based on the information obtained from the sample. Either classification of the patient is considered part of the invention because it is useful not only to determine which patients are likely to respond to a particular treatment but also to determine which patients are not likely to respond to a particular treatment. In the latter, the patient is spared from treatment with a pharmaceutical agent that is not likely to help him or her.

The sample from the cancer may be obtained by biopsy of the patient's cancer. In certain embodiments, more than one sample from the patient's tumor is obtained in order to acquire a representative sample of cells for further study. For example, a patient with breast cancer may have a needle biopsy to obtain a sample of cancer cells. Several biopsies of the tumor may be used to obtain a sample of cancer cells. In other embodiments, the sample may be obtained from surgical excision of the tumor. In this case, one or more samples may be taken from the excised tumor for further study. If the cancer is a leukemia, a sample of cancer cells may be obtained by obtaining a blood sample or bone marrow biopsy.

After the sample is obtained, it may be further processed. The cancer cells may be cultured, washed, or otherwise selected to remove normal tissue. The cells may be trypsinized to remove the cells from the tumor sample. The cells may be sorted by fluorescence activated cell sorting (FACS) or other cell sorting technique. The cells may be cultured to obtain a greater number of cells for study. In certain instances the cells may be immortalized. In addition, the cells may be frozen. The cells may be embedded in paraffin.

After the sample from the patient's cancer has been obtained, the step of determining whether a particular tubulin isotype or microtubule-associated biomolecule is present in the cells may use any technique known in the art for determining the expression, expression levels, presence of a gene product, or activity of a gene product (e.g., messenger RNA (mRNA), protein, protein complex, post-translationally modified protein, etc.). In certain embodiments, the mRNA is isolated from the cancer cell sample to determine the expression of genes of interest. In certain embodiments, the expression levels of multiple genes at once may be determined. For example, the expression of multiple isotypes of tubulin, multiple microtubule-associated biomolecules, or combinations thereof may be determined. In certain embodiments, the expression of 1, 2, 3, 4, 5, 6, 7, 8, 9, 10, 15, 20, 25, or 30 genes may be determined. The levels of mRNA transcript from a particular gene may be determined qualitatively or quantitatively using any methods known in the art. The levels of mRNA may be quantitated by quantitative PCR of the reverse transcribed RNA. The levels of mRNA may also be quantitated by Northern blot analysis. The presence of mRNA transcript may also be determined by gene chip analysis. The use of gene chips is particularly useful in determining the expression levels of multiple genes. For example, in determining the levels of expression of 10-20 or fewer genes, quantitative PCR may be used. When the expression levels of more genes are determined, gene chips are more convenient although quantitative PCR could still be used.

In certain embodiments in which gene chips are used, the gene chip may contain sequences from a variety of ESTs or the sequences may be limited to those involved in microtubule assembly. In certain embodiments, the gene chip microarray contains at least 100, 500, 1000, 10000, 15000, 20000, 25000, 30000, 35000, 40000, 45000, 50000, or 100000 sequences. The mRNA from the sample obtained from the patient is allowed to hybridize with the sequences on the microarray in order to determine the expression pattern of the genes represented on the microarray. These microarrays may be purchased from companies such as Agilent Technologies, Affymetrix, Inc., etc. In some instances, the microarray will be prepared by the researchers such as when only a subset of genes will be analyzed for expression (e.g., genes involved in microtubule assembly).

In other embodiments, rather than determining the presence of an mRNA transcript for a gene of interest, the presence or levels of the actual protein is determined. The analysis for protein may be performed using any method known in the art. In certain embodiments, antibodies directed to the protein are used. These antibodies are preferably specific for the protein of interest. In certain embodiments, the antibodies only react with one tubulin isotype or microtubule-associated protein. The antibodies may be contacted with the cancer cells directly, or the antibodies may be used in Western analysis after polyacrylamide gel electrophoresis of the proteins of the cell. These antibodies may be modified to visualize their binding to the protein of interest. For example, the antibodies may be derivatized with a fluorescence marker, the antibodies may be radiolabelled, or the antibodies may be conjugated to an enzyme such as alkaline phospatase.

The protein of interest may also be determined by mass spectroscopy. Matrix-assisted laser desorption/ionization time-of-flight (MALDI-TOF) mass spectroscopy has been used previously to determine the presence of particular proteins in a sample. MALDI-TOF spectroscopy has even been used to determine the presence of isotypes of tubulin in breast cancer cells (Verdier-Pinard et al. Biochemistry 42:5349-5357, 2003; incorporated herein by reference). Liquid chromatography-mass spectroscopy may also be used to determine the presence of particular proteins of interest in a cell sample (Verdier-Pinard et al. Biochemistry 42:12019-12027, 2003; incorporated herein by reference). The analysis of proteins of interest in cells by mass spectroscopy is based on differing ratios of m/z for the proteins being analyzed. For example, in analyzing for different isotypes of tubulin, the ratio of m/z for each isotype of tubulin must be unique in order to discern the individual isotypes from each other. In certain embodiments, the protein of interest is digested in order to analyze only a portion of the protein by mass spectroscopy. In other embodiments, the protein is partially purified. For example, tubulin may be purified away from other cellular proteins in order to better determine the tubulin isotype present in the cell. Traditional column chromatography as well as HPLC may be used to purify the protein being analyzed by mass spectroscopy.

### Tubulin Isotypes

In certain embodiments of the present invention, one or more isotypes of tubulin expressed in the cancer cells obtained from the patient is/are determined. In certain embodiments, a particular cancer cell may express only one type of each of α-tubulins and β-tubulins. More commonly, the cell will express multiple isotypes, typically at different levels. In other embodiments, different cells within the population of cancer cells will express the same or different isotypes. In humans, microtubules are composed of repeating hetereodimers of α-tubulin and β-tubulin. Microtubules are involved in many cellular functions including motility, morphogenesis, intracellular trafficking, cell shape, mitosis, and meiosis (Desai et al. Annu. Rev. Cell Dev. Biol. 13:83-117, 1997; Oakley Trends Cell Biol. 10:537-542, 2000; Sharp et al. Nature 407:41-47, 2000; each of which is incorporated herein by reference).

Both α-tubulin and β-tubulin exist as multiple isotypes. The various isotypes are each approximately 450 amino acids long. Although the isotypes are highly conserved, they display extensive sequence variations at their C-termini. The C-termini has been found to participate in binding with microtubule-associated proteins (MAPs) to microtubules (Verdier-Pinard et al. Biochemistry 42:12019-12027, 2003; Luduena Int. Rev. Cytol. 178:207-275, 1998; each of which is incorporated herein by reference). These isotypes frequently exhibit tissue-specific expression (for reviews, see Sulivan Annu. Rev. Cell Biol. 4:687-716, 1988; Luduena et al. Curr. Opin. Cell Biol. 4:53-75, 1992; Leduena Mol. Biol. Cell 4:445-457, 1993; Luduena Int. Rev. Cytol. 178:207-275 (1998); Sullivan et al. Proc. Natl. Acad. Sci. USA 83:4327-4331, 1986; each of which is incorporated herein by reference). In mammalian systems such as humans, there have been identified six α-tubulins. The six α-tubulin isotypes found in humans are as follows: α1/bα1 (NCBI accession no. CAA25855); αl/Kα1 (177403, AAC31959, AAD33871); α3 (Q13748); α4 (A25873); α6 (Q9BQE3); and α8 (Q9NY65). Seven isotypes of β-tubulin have been identified in humans: βI (NCBI protein database accession no. AAD33873, P07437); βII (AAH01352, NP_{—}001060); βIII (AAH00748, NP_{—}006077); βIVa (P04350, NP_{—}006078); βIVb (P05217); βV (NP_{—}115914); and βVI (NP_{—}110400). The entries for these proteins and their protein sequences in the NCBI protein database are incorporated herein by reference.

Human Tubulin Isotypes

| | | | | |
|---|---|---|---|---|
| Isotype | | | | |
| (accession #) | Human gene | C-terminus sequence^{a} | Tissue expression | pI^{b} |

α-TUBULINS

| | | | | |
|---|---|---|---|---|
| 1 (177403) | TUBA1/k-α1 | MAALEKDYEEVGVDSVEGEGEEEGEEY | Widely expressed | 4.94 |
| | | (SEQ ID NO: 1) | | |
| 1 (CAA25855) | TUBA3/b-α1 | 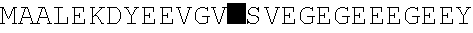 (SEQ ID NO: 2) | Mainly in brain | 5.02 |
| 3 (Q13748) | TUBA2 | 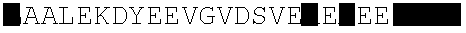 (SEQ ID NO: 3) | Testis-specific | 4.98 |
| 4 (A25873) | TUBA4 | 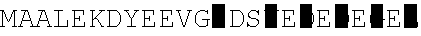 (SEQ ID NO: 4) | Brain, muscle | 4.95 |
| 6 (Q9BQE3) | TUBA6 | 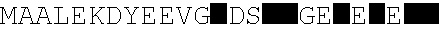 (SEQ ID NO: 5) | Widely expressed | 4.96 |
| 8 (Q9NY65) | TUBA8 | 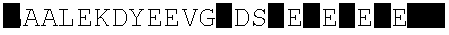 (SEQ ID NO: 6) | Heart, muscle, testis | 4.94 |

β-TUBULINS

| | | | | |
|---|---|---|---|---|
| I (AAD33873) | HM40/TUBB | YQDATAEEEEDFGEEAEEEA | Constitutive | 4.78 |
| | | (SEQ ID NO: 7) | | |
| II (AAH01352) | Hβ9/TUBB2 | 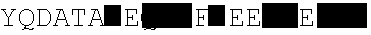 (SEQ ID NO: 8) | Major neuronal, lung | 4.78 |
| III (AAH00748) | Hβ4/TUBB4 | 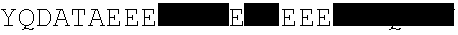 (SEQ ID NO: 9) | Minor neuronal, testis | 4.83 |
| IVa^{c}(P04350) | Hβ5/TUBB5 | 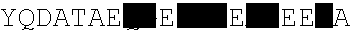 (SEQ ID NO: 10) | Brain specific | 4.81 |
| (NP_006078) | | 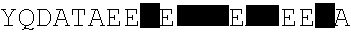 (SEQ ID NO: 11) | | 4.78 |
| IVb (P05217) | Hβ2 | 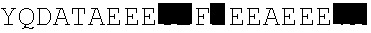 (SEQ ID NO: 12) | Major testis | 4.79 |
| V (NP_115914) | 5-beta/Beta V | 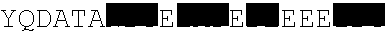 (SEQ ID NO: 13) | Uterine adenocarcinoma | 4.77 |
| VI (NP_110400) | Hβ1/TUBB1 | 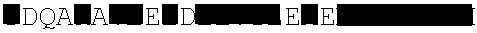 (SEQ ID NO: 14) | Blood | 5.05 |

^{a}Amino acids differeing from isotype I (Kα1) for α-tubulins or from isotype I (HM40/TUBB) for β-tubulins are highlighted in black.
^{b}The isoelectric points were calculated on the basis of the tubulin primary sequences found in the NCBI protein database (accession numbers given in first column) using the ExPaSy Compute p1/MW tool.
^{c}Two p62 IVa-tubulin sequences with distinct C-termini were found in the NCBI protein database. The top C-terminus sequence was found in human brain, and the bottom sequence was found in a human oligodendroglioma and in mouse brain.

In certain embodiments, given the sequence variations found in the C-termini of the various tubulin isotypes, the determination of whether an isotype is expressed in a cancer cell is based on PCR primers, polynucleotide probes, or peptides from the C-termini of tubulin. Antibodies used in identifying the various isotypes may be directed to the C-termini of tubulin isotypes. By focusing on the C-termini of the isotypes, the primers, probes, peptides, or antibodies are more likely to be specific for a particular isotype and not cross-react with other isotypes. In certain embodiments, the last 100, 75, 50, 40, 30, 25, 20, 15, or 10 amino acids are used in determining whether a particular tubulin isotype is expressed in the cancer cell. In certain embodiments, the last 15-25 or 15-20 amino acids of the C-terminus are used.

In addition, the tubulins undergo numerous post-translation modifications. These modification include tyrosination-detyronsination, acetylation, phosphorylation, polyglutamylation, and polyglycylation. The post-translation modification of a tubulin protein may depend on its isotype. For example, α-tubulin has been found to be acetylated and undergo tyrosination-detyronsination. βIII-tubulin has been shown to be phosphorylated. One or more such post-translational modifications may be used to determine the isotype(s) of tubulin expressed in the cancer cells of the patient.

In certain embodiments, the method of selecting patients is based on determining the α-tubulin isotype(s) expression levels or protein levels. In other embodiments, the method is based on determining the β-tubulin isotype(s) expression levels or protein levels. In certain particular embodiments, the method may focus on one, two, three, four, five, six, seven, eight, nine, ten, eleven, twelve, or thirteen particular isotypes of α- and/or β-tubulins. In certain embodiments, the method may be based on expression levels (including absence of expression) of isotype I Kα1-tubulin. In certain other embodiments, the method may be based on expression levels (including absence of expression) of isotype I (bα1) α-tubulin. In other embodiments, the method may be based on expression levels (including absence of expression) of α3-tubulin. In yet other embodiments, the method may be based on expression levels (including absence of expression) of α4-tubulin. In certain embodiments, the method may be based on expression levels (including absence of expression) of α6-tubulin. In certain embodiments, the method may be based on expression levels (including absence of expression) of α8-tubulin. In certain particular embodiments, the method is based on the expression levels of isotype I (bα1) α-tubulin (TUBA3) and α6-tubulin (TUBA6).

In certain embodiments, the method is based on determining the expression levels (including the absence of expression) of β-tubulin isotypes. For example, the method of selecting a patient may be based on the expression level (including absence of expression) of βIII-tubulin in certain embodiments. In other embodiments, the method may be based on expression levels (including absence of expression) of βI-tubulin (TUBB). In other embodiments, the method may be based on expression levels (including absence of expression) of βII-tubulin (TUBB2). In other embodiments, the method may be based on expression levels (including absence of expression) of βIVa-tubulin (TUBB5). In other embodiments, the method may be based on expression levels (including absence of expression) of βIVb-tubulin (Hβ2). In other embodiments, the method may be based on expression levels (including absence of expression) of βV-tubulin (Beta V). In other embodiments, the method may be based on expression levels of βVI-tubulin. As would be appreciated by one of skill in this art, various combinations of βI-tubulin (TUBB), βIII-tubulin (TUBB4), βIVa-tubulin (TUBB5), βVb-tubulin (Hβ2), βV-tubulin (Beta V), and βVI-tubulin (TUBB1) may be used to determine whether a patient is a candidate for a particular cancer treatment. In certain embodiments, the expression levels or protein levels of two, three, or four of βI-tubulin (TUBB), βIII-tubulin (TUBB4), βIVa-tubulin (TUBB5), βIVb-tubulin (Hβ2), βV-tubulin (Beta V), and βVI-tubulin (TUBB1) are determined. In certain embodiments, the expression levels or protein levels of two, three, or four of βIII-tubulin (TUBB4), βIVa-tubulin (TUBB5), βIVb-tubulin (Hβ2), βV-tubulin (Beta V), and βVI-tubulin (TUBB1) are determined. In certain embodiments, the expression levels or protein levels of two, three, or four of βIII-tubulin (TUBB4), βIVb-tubulin (Hβ2), (βV-tubulin (Beta V), and βVI-tubulin (TUBB1) are determined.

As would be appreciated by one of skill in this art, in certain embodiments, various combinations of isotype I (bα1) α-tubulin (TUBA3), α6-tubulin (TUBA6), βI-tubulin (TUBB), βIII-tubulin (TUBB4), βIVa-tubulin (TUBB5), βVb-tubulin (Hβ2), βV-tubulin (Beta V), βVI-tubulin (TUBB1), and stathmin may be used to determine whether a patient is a candidate for a particular cancer treatment. In certain embodiments, the expression levels or protein levels of two, three, or four of isotype I (bα1) α-tubulin (TUBA3), α6-tubulin (TUBA6), βI-tubulin, βIII-tubulin (TUBB4), βIVa-tubulin (TUBB5), βIVb-tubulin (Hβ2), βV-tubulin (Beta V), βVI-tubulin (TUBB1), and stathmin are determined. In certain embodiments, the expression levels or protein levels of two, three, or four of isotype I (bα1) α-tubulin (TUBA3), α6-tubulin (TUBA6), βIII-tubulin (TUBB4), βIVa-tubulin (TUBB5), βIVb-tubulin (Hβ2), βV-tubulin (Beta V), βVI-tubulin (TUBB1), and stathmin are determined. In certain embodiments, the expression levels or protein levels of two, three, or four of isotype I (bα1) α-tubulin (TUBA3), α6-tubulin (TUBA6), βIII-tubulin (TUBB4), βIVb-tubulin (Hβ2), βV-tubulin (Beta V), βVI-tubulin (TUBB1), and stathmin are determined.

In certain embodiments, the expression levels or protein levels of MAP4 or Tau may be determined in combination with the groups recited above. In certain particular embodiments, the expression levels or protein levels of MAP4 may be determined in combination with the groups recited above. In certain particular embodiments, the expression levels or protein levels of Tau may be determined in combination with the groups recited above.

In certain embodiments, mutations, polymorphisms, alleles, or other forms of one or more tubulin genes is determined in identifying patients for treatment. The present invention is not limited to determining only isotypes of tubulin.

### Other Microtubule-Associated Biomolecules

In determining whether a patient is a candidate for a treatment with a particular chemical compound, not only may the tubulin isotype be used in the determination, but other microtubule-associated biomolecules may also be assessed in combination with tubulin isotypes or alone. Any biomolecules known to be directly or indirectly involved in the assembly or disassembly of microtubules may be useful in the inventive system. These biomolecules may include polynucleotides (e.g., mRNA, genes), proteins, peptides, organelles, metabolites (e.g., GTP, GDP), etc. Certain examples of biomolecules found to be associated with microtubule assembly or disassembly include centrioles, centrosomes (also known as, microtubule organizing center (MTOC)), γ-tubulin, microtubule-associated proteins (MAPs), kinases, phosphorylases, and catastrophe-promoting proteins. The invention also includes the use of other microtubule-associated biomolecules not identified at this time.

In certain embodiments, a characteristic of the centrioles found in the cancer cells of the patient is used to determine susceptibility to a particular chemical compound. Centrioles are cylindrical structures, which are typically found in pairs oriented at right angles to each other. Each cylinder is comprised of nine interconnected triplet microtubules, arranged as a pinwheel. The α- and β-tubulin heterodimers found in centriolar microtubules are post-translationally modified by polyglutamylation. Organisms that contain centrioles, such as humans, have additional tubulins. These additional tubulins are designated d, e, z, and h, and are postulated to have roles in centriole structure or assembly. In certain embodiments, isotypes or mutations in these tubulins are used to determine a patient's susceptibility to a chemical compound.

The centriole is surrounded by a mass of protein called the centrosome (also known as the microtubule organizing center). Any protein found in the centrosome may be used in the present invention to select patients for treatment. Examples of proteins that have been found in the centrosome or found to be associated with centrioles include centrin, pericentrin, ninein, and γ-tubulin. Isotypes, polymorphisms, mutations, or other forms of any protein found to be associated with the centrosome may be useful in the present invention. During cell division the centrosomes assist in organizing the mitotic spindle. The centrosome is usually located near the nucleus during interphase, and microtubules grow out from the centrosome. The microtubules grow and shrink through the addition and loss of tubulin heterodimers from their ends (plus ends). During cell division the movement of the microtubule spindles allows for the separation of duplicated chromosomes into each of the daughter cells. Drugs that target microtubule assembly interfere with microtubule spindle-mediated chromosome segregation. Typically, the drugs in this class can be divided into two categories: microtubule-stabilizing agents such as Taxol, and microtubule-destabilizing drugs such as Vinca alkaloids and colchicine. Therefore, proteins or other biomolecules that participate in this process of chromosome segregation may be useful in determining whether a patient is susceptible to treatment with such an agent. Particular isotypes, polymorphisms, mutations, alleles, or other forms of these proteins may lead to susceptibility or resistance to these agents.

In certain embodiments, the expression levels or protein levels of γ-tubulin are determined in the inventive system. γ-tubulin is homologous to α- and β-tubulins and nucleates microtubule assembly within the centrosome. Several γ-tubulin molecules associate with proteins called grips (gamma ring proteins) to form a γ-tubulin ring complex. Microtubules nucleated with the γ-tubulin ring complex appear capped at one end (the minus end). Grip proteins of the cap are thought to be involved in mediating binding to the centrosome. Phosphorylation of a conserved tyrosine residue of γ-tubulin has been shown to regulate microtubule nucleation. Various forms of γ-tubulin as well as gamma ring proteins may be assessed in selecting a patient for treatment using the inventive system. In certain embodiments, the phosphorylation of the conserved tyrosine of γ-tubulin is used in selecting a patient.

In other embodiments, the expression of microtubule-associated proteins (MAPs) is determined in the inventive system. The expression levels or protein levels of any MAP may be determined. MAPs are a diverse class of proteins that bind to microtubules. Some MAPs stabilize microtubules while other destabilize microtubules. Other MAPs cross-link adjacent microtubules. Some MAPS link microtubules to membranes or to intermediate filaments. Type I MAPs are typically found in axons and dendrites of nerve cells; however, type I MAPs have also been found in non-neural cells. Type I MAPs have repeats of the sequence KKEX (Lys-Lys-Glu-X) that bind to negatively charged tubulin domains. In certain embodiments, the protein levels or expression of a Type I MAP is determined in the inventive system. Type II MAPs such as MAP-4 and Tau are found in axons, dendrites, and non-neural cells. Type II MAPs have 3-4 repeats of an 18 amino acid sequence that binds tubulin. In certain embodiments, the protein levels or expression of a Type II MAP is determined in the inventive system. In particular embodiments, the MAP-4 expression levels or protein levels are assessed in selecting a patient for treatment. MAP-4 may be used in the inventive system in conjunction with determining tubulin isotypes in the cancer cells. In other embodiments, Tau expression levels or protein levels are determined, optionally in conjunction with tubulin isotypes. In other embodiments, the expression levels or protein levels of XMAP215 is determined. XMAP215 is a highly conserved MAP of 215 kDa and plays a role in controlling microtubule dynamics in relation to the cell cycle. XMAP215 stabilizes the plus ends of microtubules, thereby promoting the growth at the plus end and preventing catastrophic shrinkage.

In other embodiments, catastrophe-promoting proteins (catastrophins) are assessed. Catastrophe is the rapid disassembly of microtubules. Stathmin is a catastrophin that increases in abundance in some cancer cells; therefore, levels of stathmin may be determined in selecting a patient for a particular cancer therapy. Optionally, stathmin levels may be determined in conjunction with determining tubulin isotypes expressed in the patient's cancer cells. Another catastrophin is XKCM1, which is a member of the MCAK subfamily of kinesin motor proteins. XMAP215 antagonizes the effect of XKCM1. Levels of XKCM1 in the patient's cancer cells may also be determined in the selection system of the present invention.

The invention may also include determining the expression levels or protein levels of a homolog of the bacterial protein FtsZ. FtsZ is considered to be an ancestor of tubulin and has been found to play a role in bacterial cytokinesis. FtsZ can assemble into protofilaments, and the FtsZ protofilaments can assemble to form sheets or tubules. Homologs of FtsZ in higher organisms may be used in the invention to determine whether a patient is suitable for a particular cancer treatment. In other embodiments, bacterial cells expressing FtsZ may be used to identify compounds that can be used as anti-neoplastic agents such as by interfering with microtubule formation. FtsZ may be used in bacterial cells to establish a correlation between a chemical compound and susceptibility to treatment with chemical compounds that affect microtubule assembly or disassembly.

Any of the microtubule-associated biomolecules described herein may be used in selecting patients for treatment using a particular chemical compound. The determination of expression levels or protein levels of a microtubule-associated biomolecule may be performed alone in selecting patients, or the determination may be made in conjunction with other microtubule-associated biomolecules or tubulin isotypes. In certain embodiments, the expression levels or protein levels of MAP-4 are determined. In other embodiments, the expression levels or protein levels of Tau are determined. In yet other embodiments, the expression levels or protein levels of stathmin are determined. In other embodiments, the expression levels or protein levels of CLIP-170 are determined. In certain embodiments, the expression levels or protein levels of EB1 are determined. In other embodiments, the expression levels or protein levels of p150 are determined. In certain embodiments, the β-tubulin isotype found in the cancer cells is determined in conjunction with MAP-4, Tau, stathmin, CLIP-170, EB1, and/or p150. In certain other embodiments, the β-tubulin isotype levels found in the cancer cells are determined in conjunction with MAP-4 expression levels or protein levels. In yet other embodiments, the β-tubulin isotype levels found in the cancer cells are determined in conjunction with stathmin expression levels or protein levels. In still other embodiments, the β-tubulin isotype levels found in the cancer cells are determined in conjunction with CLIP-170 expression levels or protein levels. In other embodiments, the β-tubulin isotype levels found in the cancer cells are determined in conjunction with EB1 expression levels or protein levels. In certain embodiments, the β-tubulin isotype levels found in the cancer cells are determined in conjunction with p150 expression levels or protein levels.

In certain other embodiments, the expression levels or protein levels of the multidrug transporter P-glycoprotein (P-gp) is determined. Although P-gp is not involved in microtubule assembly, it is known to a play a role in resistance to cytotoxic compounds including those that affect microtubule assembly. For example, the resistance of some cancers to paclitaxel (Taxol®) has been shown to be due to the presence of the multidrug transporter P-glycoprotein (Horwitz et al. J. Natl. Cancer Inst. Monogr. 15:55-61, 1993; incorporated herein by reference). The expression levels or protein levels of P-gp may be tested in conjunction with determining tubulin isotypes or other microtubule-associated biomolecules in the sample from the patient.

### Identifying Patients

Based on the expression levels or protein levels of a particular tubulin isotype or microtubule-associated biomolecule or a combination thereof, a patient is selected for treatment using a particular chemical compound. In certain embodiments the chemical compound used to treat the patient is a compound known to interfere with microtubule assembly/disassembly. In certain embodiments, the compound binds to α-tubulin. In other embodiments, the compound binds to β-tubulin. In certain embodiments, the chemical compound is an organic compound. In certain embodiments, the chemical compound is a small molecule. In certain embodiments, the compounds have anti-neoplastic activity. The compound may be approved by the FDA for use in humans or may be undergoing review by the FDA for use in humans.

In certain particular embodiments, the compound is a halichondrin B analog. In certain embodiments the compound is a halichondrin B analog having anticancer and/or anti-mitotic activity. Preferably, the halichondrin B analog is an anti-microtubule agent, which interferes with the assembly or disassembly of microtubules. In certain embodiments, the analogs have the formula (I):

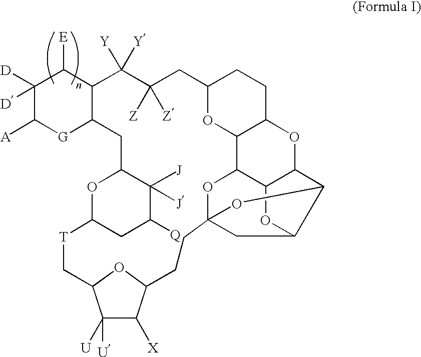

wherein A is a C₁₋₆ saturated or C₂₋₆ unsaturated hydrocarbon skeleton, the skeleton being unsubstituted or having between 1 and 13 substituents, preferably between 1 and 10 substituents, e.g., at least one substituent selected from cyano, halo, azido, Q₁, and oxo, wherein each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁ and O(CO)NR₂R₁, and the number of substituents can be, for example, between 1 and 6, 1 and 8, 2 and 5, or 1 and 4;

wherein each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl (e.g., p-fluorophenyl or p-chlorophenyl), C₆₋₁₀ hydroxyaryl, C₁₋₄ alkoxy-C₆ aryl (e.g., p-methoxyphenyl, 3,4,5-trimethoxyphenyl, p-ethoxyphenyl, or 3,5-diethoxyphenyl), C₆₋₁₀ aryl-C₁₋₆ alkyl (e.g., benzyl or phenethyl), C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl;

wherein each of D and D′ is independently selected from R₃ and OR₃, wherein R₃ is H, C₁₋₃ alkyl, or C₁₋₃ haloalkyl;

wherein the value for n is 1 or preferably 0, thereby forming either a six-membered or five-membered ring, wherein the ring can be unsubstituted or substituted, where E is —R₅ or —OR₅, and can be a heterocyclic radical or a cycloalkyl, e.g., where G is S, SH₂, NR₆, or preferably O;

wherein each of J and J′ is independently H, C₁₋₆ alkoxy, or C₁₋₆ alkyl; or J and J¹ taken together are ═CH₂ or —O-(straight or branched C₁₋₅alkylene or alkylidene)-O—, such as exocyclic methylidene, isopropylidene, methylene, or ethylene;

wherein Q is C₁₋₃ alkyl, and is preferably methyl;

wherein T is methylene, ethylene, or ethenylene, optionally substituted with (CO)OR₇, where R₇ is H or C₁₋₆ alkyl;

wherein each of U and U′ is independently H, C₁₋₆ alkoxy, or C₁₋₆ alkyl; or U and U′ taken together are ═CH₂ or —O-(straight or branched C₁₋₅alkylene or alkylidene)-O—;

wherein X is H or C₁₋₆alkoxy;

wherein each of Y and Y′ is independently H or C₁₋₆ alkoxy; or Y and Y′ taken together are ═O, ═CH₂, or —O-(straight or branched C₁₋₅alkylene or alkylidene)-O—;

wherein each of Z and Z′ is independently H or C₁₋₆ alkoxy; or Z and Z′ taken together are ═O, ═CH₂, or —O-(straight or branched C₁₋₅alkylene or alkylidene)-O—.

In certain embodiments, the halichondrin B analog has the stereochemistry as shown in the formula (II):

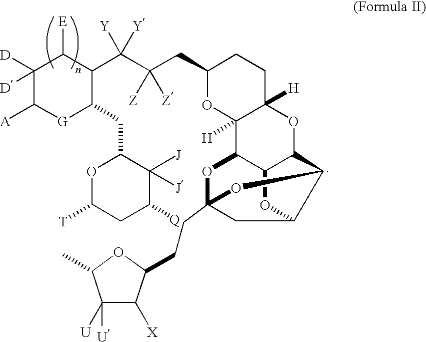

In certain embodiments, Q is methyl. In certain embodiments, J and J′ taken together are ═CH₂. In certain embodiments Z and Z′ taken together are ═O. In certain embodiments, Y is hydrogen. In certain embodiments, Y′ is hydrogen. In certain embodiments, T is ethylene. In certain embodiments, G is oxygen. In certain embodiments, n is zero, and E is absent. In certain embodiments, D is methoxy, and D′ is hydrogen. In other embodiments, D is hydrogen, and D′ is methoxy.

In certain embodiments, the halichondrin B analog is of the formula (III):

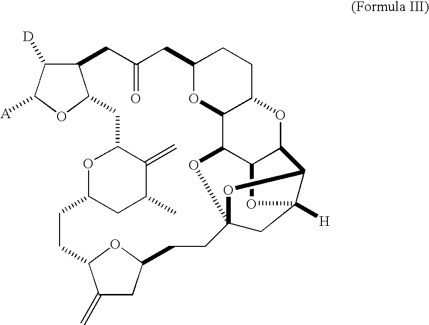

wherein A and D are as defined above.

In certain embodiments, the halichondrin B analog is of the formula (IV):

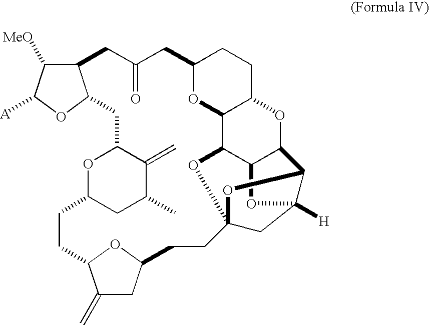

wherein A is as defined above.

In certain embodiments, A is a C₁₋₆ saturated or C₂₋₆ unsaturated hydrocarbon skeleton having at least one substituent selected from the group consisting of cyano, halo, azido, oxo, amino, and hydroxyl. In certain other embodiments, A is a C₁₋₆ saturated or C₂₋₆ unsaturated hydrocarbon skeleton having at least one substituent selected from the group consisting of amino, azido, and hydroxyl. In other embodiments, A is a C₁₋₆ saturated or C₂₋₆ unsaturated hydrocarbon skeleton having at least two substituents selected from the group consisting of amino and hydroxyl. In other embodiments, A is a C₁₋₆ saturated or C₂₋₆ unsaturated hydrocarbon skeleton having at least one hydroxyl substituent and at least one amino substituent. In other embodiments, A is a C₁₋₆ saturated or C₂₋₆ unsaturated hydrocarbon skeleton having at least one hydroxyl substituent and at least one cyano substituent. In other embodiments, A is a C₁₋₆ saturated or C₂₋₆ unsaturated hydrocarbon skeleton having at least two hydroxyl substituents. In other embodiments, A comprises a C₂₋₄ hydrocarbon skeleton. In yet other embodiments, A comprises a C₃ hydrocarbon skeleton. In certain embodiments, A is A is a C₁₋₆ saturated or C₂₋₆ unsaturated hydrocarbon skeleton, the skeleton being unsubstituted or having between 1 and 4 substituents selected from the group consisting of azido, hydroxy, OR₁, NH₂, NR₁R₂, NR₂(CO)R₁, NR₂(CO)(CO)R₁, NR₄(CO)NR₂R₁, and NR₂(CO)OR₁; wherein each of R₁, R₂, and R₄ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₄ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl.

In certain embodiments, the analog has the formula (V):

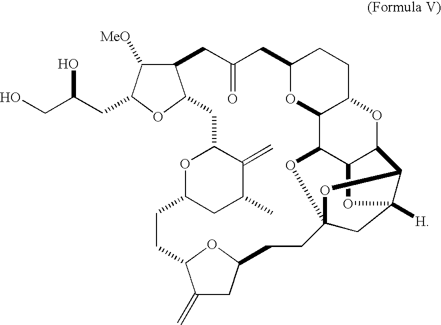

In certain embodiments, the analog is E7389 which has the formula (VI):

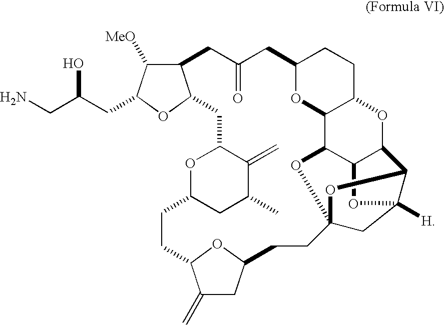

Halichondrin B analogs, the synthesis, methods of treatment, and pharmaceutical compositions thereof are described in U.S. Pat. Nos. 6,214,865; 6,365,759; 6,469,182; and 6,653,341; each of which is incorporated herein by reference; and U.S. patent applications, U.S. Ser. No. 60/576,642, filed Jun. 3, 2004; U.S. Ser. No. 60/626,769, filed Nov. 10, 2004; and U.S. Ser. No. 10/687,526, filed Oct. 16, 2003; each of which is incorporated herein by reference.

In certain embodiments, a halichondrin analog or a pharmaceutical composition thereof is used to treat the cancer patient if the cells of the cancer are found to express class III isotype β-tubulin at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express class IVb isotype β-tubulin at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express class V isotype β-tubulin at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express class VI isotype β-tubulin at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express class 1 isotype α-tubulin (TUBA3/b-β1) at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express class 6 isotype α-tubulin (TUBA6) at elevated levels (e.g., at least 2, 3, 4, or 5 times the level observed in control cells). In other embodiments, a halichondrin B analog or a pharmaceutical composition thereof is used to treat the cancer patient if the cells of the cancer are found to express stathmin at elevated levels (e.g., at least 2, 3, 4, or 5 times the level observed in control cells). In certain embodiments, the compound to be used in the treatment is a member of the genus or sub-genuses of halichondrin analogs as described herein. In a particular embodiment, the halichondrin B analog used to treat the selected patient is E7389.

After the patient has been selected for treatment using a particular chemical compound, the patient may be treated by the administration of the compound or a pharmaceutical composition thereof in a therapeutically effective amount. The treatment may include multiple administrations of the compound or a pharmaceutical composition thereof over weeks or months. In certain embodiments, the compound is a halichondrin B analog such as E7389 as described herein. The dosing of the compound may range from 0.001 mg/m² to 100 mg/m², or 0.001 mg/m² to 10 mg/m², or 0.01 mg/m² to 10 mg/m², or 0.1 mg/m² to 75 mg/m², or 1 mg/m² to 50 mg/m².

### Determining Correlations Between Chemical Compounds and Gene Expression

In light of the correlations between the halichondrin B analog E7389 and the hemiasterlin analog E7974 and the expression of tubulin isotypes or microtubule-associated biomolecules as established by the inventors, those of ordinary skill in this art will appreciate that correlations for other compounds and other markers can be determined. Such correlation will find use in the above-described methods of identifying and treating patients. Such correlations will offer cancer patients better, more effective treatment. In certain embodiments, the chemical compounds used in establishing the correlation are anti-microtubule agents (i.e., agents which interfere with the assembly or disassembly of microtubules in the cell). In certain embodiments, the compounds will bind microtubules, or α-tubulin, or β-tubulin. In certain other embodiments, the compounds are halichondrin B analogs as described herein.

In the inventive system, cells are exposed to the test compound for a defined period of time. The inhibition of growth or other phenotype is then determined for the cells contacted with the test compound. The tubulin isotype expression or expression of other microtubule-associated proteins is determined for the cells contacted with the test compound. These data are then used to calculate correlations between the test compound and the expression of tubulin isotype or microtubule-associated proteins. A p-value of 0.05 or less is typically considered statistically significant; however, in certain embodiments, p-values of 0.07 or less, 0.10 or less, 0.15 or less, or 0.20 or less are considered significant. Greater p-values may be accepted when a lesser number of cell lines are tested in the inventive system.

The cells used in the inventive system may be obtained from any source. Preferably, the cells can be grown reliably and reproducibly in cell culture. The cells may be from any species including bacteria, fungi, mammalian, human, yeast, rat, mouse, E. coli, S. cerevisiae, etc. When the cells are from higher organisms, the cells may be derived from any tissue, e.g., neural, brain, skin, muscle, endocrine, lung, heart, stomach, colon, liver, kidney, pancreas, bladder, breast, ovarian, testicular, prostate, blood, bone marrow, bone, connective tissue, thyroid, adrenal, pituitary gland, spleen, etc. In certain embodiments, the cells may be of endodermal, mesodermal, or ectodermal origin. In certain embodiments, the cells may be cancer cells. In other embodiments, the cells are immortalized. The cells may be derived from a biopsy of a patient with cancer. The cells may also be obtained from a surgical specimen. The cells may also be obtained from the blood of a patient.

In certain embodiments, the cells are obtained from a commercial source or a depository of cell lines (e.g., ATCC or an equivalent foreign depository). The cells may also be obtained from the NCI-Anticancer Drug Screen Panel. In certain embodiments, the cells are breast cancer cell lines. Examples of breast cancer cell lines are AU565, BT-20, MCF-7, MDA-MB-231, MDA-MB-435, MDA-MB-468, HCC38, HCC70, HCC1143, HCC1419, HCC1428, HCC1500, HCC1599, HCC1806, HCC1954, HCC2218, UACC-812, UACC-893, ZR-75-1, HS 578T, and ZR-75-30. In other embodiments, the cells are lung cancer cell lines. Examples of lung cancer cell lines include NCI-H460, A549, A549-T12, NCI-H460, and A549-T24. In yet other embodiments, the cells are ovarian cancer cell lines. Ovarian cell lines include OVCAR-3 and IGROV1. Drug-resistant cell lines or sub-lines may also be used in the present invention. For example, the cell line may be resistant to other anti-microtubule agents such as taxol, Vinca alkaloids, taxotere, etc. In certain embodiments, at least 5, 10, 15, 20, 25, 30, or 50 cell lines are used. As would be appreciated by one of skill in this art, as the number of cell lines increases, the correlations established become more significant. Preferably, approximately 20 cell lines are used in the inventive system.

The cells are contacted with a test compound. The test compound may be an anti-microtubule agent. The test compound may be a halichondrin B analog as described herein. Various concentrations of the test compound may be used ranging from 0.001 μM to 100 mM, or 0.01 μM to 10 mM, or 0.01 μM to 1 mM, 0.1 μM to 1 mM. The test compound is contacted with the cells over hours, days, or weeks. In certain embodiments, the test compound is contacted with the cells for 1-14 days. In other embodiments, the test compound is contacted with the cells for 2-10 days. In other embodiments, the test compound is contacted with the cells for approximately 7 days. In other embodiments, the test compound is contacted with the cells for approximately 4 days.

At the end of the test period, a phenotype of the cells is assessed using methods known in the art. For example, a cell growth inhibition assay may be used. Cell growth may be assessed using a modified methylene blue-based microculture assay (Amin et al. Cancer Res. 47:6040-45, 1987; Finlay et al. Anal. Biochem. 139:272-277, 1984; each of which is incorporated herein by reference). Other aspects or characteristics of the cells exposed to the test compound may also be assessed such as size of cell, cell death, number of cells undergoing mitosis, mitotic spindles, cells in S phase of the cell cycle, etc.

As described above in the patient selection system, the expression levels or protein levels of tubulin isotypes or microtubule-associated proteins is then determined for the cells tested. Any methods can be used for determining expression levels or protein levels including Northern blot, PCR techniques, Western blot, mass spectroscopy, immunoassay, immunoassay, staining of the cells, etc. Once the expression levels or protein levels for the proteins of interest are determined, this data may be correlated with the phenotype data (e.g., IC₅₀s obtained from cell growth inhibition assays) using statistical methods.

In certain embodiments, the standard comparative C_{T} method for relative quantitation of gene expression is used. Gene expression levels may be normalized to levels of expression of a housekeeping gene such as GAPDH. A baseline of expression of the gene of interest may also be established in a control cell line. In certain embodiments, a conventional threshold of correlation coefficient (Pearson r) is considered significant with a p-value of 0.05 or less. P-values of 0.20 or less, 0.15 or less, or 0.10 or less may also be used. Greater p-values may be particularly useful when the number of cell lines is less than 20. In certain embodiments, the experiments using a particular test compound will be repeated with a greater number of cell lines to establish statistically significant correlations.

Once a statistically significant correlation has been established, the correlation can then be used in selecting patients for treatment with the test compound or compounds related to the test compound. The inventive system for identifying and/or treating patients is described above. In certain embodiments, the correlation may be based on a particular level or range of a tubulin isotype or microtubule-associated biomolecule. For example, a particular protein range in the cancer cells, or a particular range of mRNA levels in the cancer cells may be used to establish a correlation. In other embodiments, more than one marker may be determined in order to establish a statistically significant correlation. In certain embodiments, two, three, four, five, or more markers may need to be analyzed to establish statistical significance. Again, the presence or absence of a marker may be used, or the level or range of a marker may be used, or a combination thereof.

In certain embodiments, multiple test compounds are tested. In certain instances, there may be correlations between multiple test compounds. For example, E7974, E7389, and vinblastine were found to correlate in the panel of cell lines described in Example 1. These compounds did not correlate with paclitaxel. Such results may indicate that E7974, E7389, and vinblastine may be useful in treating the same cancer and that these compounds may be useful in treating cancers that are not susceptible to treatment with paclitaxel.

A similar method may be used to identify chemical compounds that are effective in treating cancers expressing a particular tubulin isotype or microtubule-associated biomolecule. The method is particularly useful in identifying compounds useful in the treatment of cancer refractory to other known treatments, for example, breast cancer not susceptible to treatment with paclitaxel (Taxol®). In this method, libraries or collections of chemical compounds are screened to identify those compounds, which inhibit cell growth and the inhibition correlates with expression levels or protein levels of a particular tubulin isotype or microtubule-associated biomolecule. The identified compounds may serve as a lead compound or as a drug candidate.

### Kits

Kits for a clinicians or researchers practicing the claimed methods may include the materials, reagents, equipment, and instructions conveniently packaged for use. The kits may include polynucleotides (e.g., primers, PCR primers, probes, DNA, RNA, DNA analogs, etc.), buffers, enzymes (e.g., ligases, endonucleases, phosphatases, kinases, proteases, polymerase, heat-stable polymerases, etc.), Eppendorf tubes, instruction manuals, nucleotides, chromatography materials (e.g., gel filtration, ion exchange, size exclusion), spin columns, test compounds (e.g., halichondrin B analogs, paclitaxel (Taxol®), taxotere, colchicine, vinblastine, nocodazole, other anti-microtubule agents), cell lines (cancer cell lines, breast cancer cell lines, lung cancer cells lines, ovarian cancer cells lines), growth media, solvent (e.g., DMSO, DMF).

The kits useful in practicing the method of selecting patients may include equipment and materials useful in obtaining a sample of the patient's cancer. Such equipment and materials may include syringes, needles, scalpels, cups, tubes, labels, etc. The kits may also contain materials for purifying mRNA from the sample when the tubulin isotype or microtubule-associated protein expression is determined by gene chip, Northern blot, or PCR. When immunoassays are used in the claimed method, antibodies directed to the proteins whose expression is to be determined are included in the kit. Preferably, the antibodies are specific for a marker.

The kits useful in establishing correlations between markers and test compounds may include cell lines, control compounds, statistical software, growth media, buffers, solvents for dissolving the test and control compounds, tissue culture plates (e.g., 96-well plates), materials for conducting a growth inhibition assay, and material needed for detecting the expression levels or protein levels of tubulin isotypes of microtubule-associated biomolecules. Preferably, the kit includes all a researcher would need for establishing correlations as described herein except for the test compounds. The test compounds are typically supplied by the researcher. Particular cells lines may also be provided by the researcher.

The present invention also includes reagents used in practicing the claimed methods. These reagents include primers, probes, or antibodies specific to tubulin isotypes or microtubule-associated biomolecules. Example of primers and probes useful in the practice of the invention are listed in Table 1 and 2 of the Examples.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Establishing a Correlation between Tubulin Isotypes and E7389 and E7974

### Material and Methods

### Cell Lines

The following human breast cancer cell lines were obtained from the ATCC. Cells were maintained according to ATCC-recommended culture conditions. AU565 (ATCC Catalog No. ATCC Catalog No. CRL-2351), BT-20 (ATCC Catalog No. HTB-19), MCF7 (ATCC Catalog No. HTB22), MDA-MB-231 (ATCC Catalog No. HTB-26), MDA-MB-435 (ATCC Catalog No. HTB-129), MDA-MB-468 (ATCC Catalog No. HTB-132), HCC38 (ATCC Catalog No. CRL-2314), HCC70 (ATCC Catalog No. CRL-2315), HCC1143 (ATCC Catalog No. CRL-2321), HCC1428 (ATCC Catalog No. CRL-2327), HCC1500 (ATCC Catalog No. CRL-2329), HCC1806 (ATCC Catalog No. CRL-2335), HCC1954 (ATCC Catalog No. CRL-2338), HCC2218 (ATCC Catalog No. CRL-2343), UACC-812 (ATCC Catalog No. CRL-1897), UACC-893 (ATCC Catalog No. CRL-1902), ZR-75-1 (ATCC Catalog No. CRL-1500), ZR-75-30 (ATCC Catalog No. CRL-1504).

### Cell Growth Inhibition Assay

Cultured human breast cancer cells were placed in 96-well plates and grown in the continuous presence of test compounds for 4 or 7 days. To expose the cells to test compounds for 7 days, medium was replaced with fresh medium containing compounds after 4 days of exposure and cells were incubated for 3 additional days. Cell growth was assessed using modifications (Amin et al., Cancer Res., 47:6040-6045, 1987; incorporated herein by reference) of a methylene blue-based microculture assay (Finlay et al., Anal. Biochem., 139:272-277, 1984; incorporated herein by reference).

To investigate the possible expression of PgP by the cell lines used in this study, the antiproliferative effects of paclitaxel were determined in the presence and absence of verapamil, a known inhibitor of PgP.

RNA Isolation and cDNA Synthesis

For total cellular RNA isolation, cells were harvested by trypsinization. Cells were washed twice with phosphate-buffered saline (PBS). RNAlater RNA Stabilization Reagent (Qiagen) was added to cell pellets and samples were stored at −80° C. until RNA isolation. RNeasy Protect Mini Kit (Qiagen, cat.no. 74124) was used to isolate total RNA from cells. Standard manufacturer protocol was used in this procedure. QIAshredder spin columns (Qiagen cat. no. 79654) were used to homogenize samples and on-column DNase digestion step (RNase-Free DNase Set, Qiagen, cat. no. 79254) was included to remove any DNA contamination.

About 1-2 ug of total cellular RNA were then used in a RT-PCR reaction for cDNA synthesis using RETROscript™ Kit (Ambion, cat. no. 1710). The reaction was carried out according to provided manufacturer's protocol. Equal mixtures of Oligo(dT) and random decamer primers were used in the reaction.

### Primers and Probes, Quantitative Real-Time PCR

### Beta-Tubulin Genes

Seven beta tubulin genes (Class I isotype, gene HM40/TUBB; Class II isotype, gene Hb9/TUBB2; Class III isotype, gene Hb4/TUBB4; Class IVa isotype, gene Hb5/TUBB5; Class IVb isotype, gene Hb2; Class V isotype, gene 5-beta/Beta V; and Class VI isotype, gene Hb1/TUBB1) are highly homologous to each other in the 5′-end region. For this reason we have chosen amplicons from 3′-end of each gene where there homology is low. Sequences of gene-specific primers and probes are presented in Table 1. Probes were labeled by FAM reporter and TAMRA quencher. A total of 1 uL of the synthesized cDNA served as a substrate for a PCR amplification of each gene of interest. Quantitative real-time PCR was performed in 96-well plates using gene specific primers and probes with ABI PRISM 7700 Sequence Detection System (Applied Biosystems, Foster City, Calif.). Each sample was assayed in triplicate using TaqMan Universal PCR Master Mix (Applied Biosystems, cat. no. 4304437). Manufacturer's suggested thermal cycling conditions were used at the annealing temperature of 59° C.

### Stathmin, MAP4, Tau, and Alpha-Tubulin Genes

Forward and reverse primers and probes for stathmin and MAP4 were designed from the 3′-end of the genes are shown in Table 1. Specific probes and primers for Tau and six alpha tubulin genes were received from Applied Biosystems (Table 2). Quantitative analysis of stathmin and MAP4 mRNA was performed using Taqman One-Step RT-PCR Master Mix Reagents Kit (Applied Biosystems, cat. no. 4309169). Standard thermal cycling parameters were used with a 30 min incubation at 48° C. and a 10 min incubation at 95° C. followed by 40 cycles of a 15 sec incubation at 95° C. and a 1 min incubation at 60° C. The expression of alpha-tubulin isotypes was analyzed in the same way as beta-tubulin isotypes as described above.

### Calculation of Relative Expression Level of Genes and Statistical Analysis

The standard comparative C_{T} method for relative quantitation of gene expression was used (ABI tutorial). Gene expression levels were normalized to levels of expression of the GAPDH control. Expression level of gene of interest in AU565 cell line was chosen as a reference (baseline) control for comparisons.

Correlations between sensitivity of cell lines to test agents (IC₅₀s obtained in the cell growth inhibition assays) and the expression level of genes of interest were calculated. A conventional threshold of correlation coefficient (Pearson r) was considered significant with a p-value of 0.05 or less.

Standard multiple stepwise regression analysis was performed on obtained data. For this analysis the IC₅₀ values of the four compounds were standardized, and the fourteen gene expressions were subjected to both standardization and log10 transformation in all of the analyses.

### Results

Antiproliferative effects of four tubulin-binding anticancer agents were evaluated by a methylene blue-based cell growth inhibin assay in a panel of 19 human breast cancer cell lines. The following agents were used in this study: halichondrin analog E7389, hemiasterlin analog E7974, vinblastine, and paclitaxel. Each IC₅₀ determination was conducted in at least three separate experiments. A presence of multidrug resistance efflux pump (P-glycoprotein or PgP) could markedly affect sensitivity of cell lines to test agents and thus mask correlations between beta-tubulin expression and cell lines' sensitivity to test agents. Cell lines were therefore tested for evidence of PgP expression by monitoring effects of the PgP blocker verapamil (used at a 10 βM concentration) on paclitaxel sensitivity. No evidence for PgP expression was found in any of the cell lines tested (Table 4). The average IC₅₀ values for cell growth inhibition are shown in Table 3. E7389 was the most active of the four compounds, inhibiting growth of breast cancer cell lines with IC₅₀ values ranging from 0.29 to 1.8 nM. A fold-difference between IC₅₀'s in the most sensitive and the least sensitive cell line was approximately the same for all four compounds (6.2, 6.8, 5.8, and 5.6 for E7389, E7974, paclitaxel, and vinblastine, respectively). Inter-drug correlations were seen between sensitivities to the three microtubule polymerization inhibitors E7974, E7389, and vinblastine, but not between the microtubule polymerization stabilizer paclitaxel.

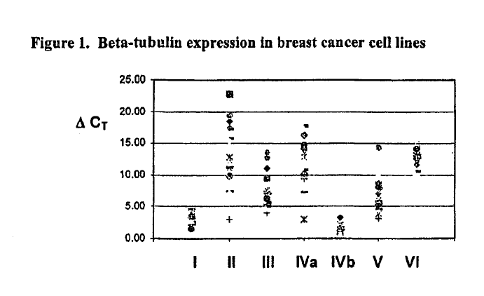

Expression analysis of seven beta-tubulin isotype genes, four alpha-tubulin isotype genes, as well as stathmin, Tau, and MAP4 genes was examined by quantitative real-time PCR in 19 human breast cancer cell lines. Normalization of each gene expression to GAPDH mRNA was done in the each experiment. Based on C_{T} data, the most highly expressed genes among beta-tubulins were the Class I and Class IVb isotypes. The beta tubulin genes expressed at the lowest levels in the panel of cell lines tested in this study were Class II, Class IVa and Class VI isotypes. To compare gene expression among the cell lines, the level of transcripts in cell line AU565 was chosen arbitrarily as a baseline (Table 5). Most varied gene expression levels among cell lines were observed for Class II, Class III and Class IVa beta-tubulin isotypes (FIG. 1).

Comparison of cell line's sensitivity to tubulin-binding agents with expression level of nine studied genes was performed first using correlation analysis (Table 6). The highest correlation among beta-tubulin genes with the effect of all four compounds was with Class III isotypes. This correlation reached significant levels in cases of E7389 and E7974. Interestingly, the observed correlations were negative, meaning that the higher expression level of Class III isotype associated with higher sensitivity to E7389 and E7974. E7974 also showed some correlation with the expression of Class I isotype, approaching significant level (r=−0.42, p=0.07).

Although the expression level of stathmin and MAP4 genes were not significantly correlated with compound's effect on cell growth in this type of analysis, correlations with E7974 were the highest and may become meaningful with the inclusion of more cell lines in the analysis.

Association of selected gene expression levels and sensitivity to tubulin-binding agents was further evaluated using standard multiple stepwise regression analysis (Table 7). IC₅₀ of E7389 significantly correlated with the expression of Class III, IVb, and V beta-tubulin isotypes, as well as Class I alpha-tubulin isotype. Sensitivity to E7974 was associated again with Class III and IVb beta-tubulin isotypes, but also with Tau and stathmin gene expression in this analysis.

TABLE 1

Beta-tubulin isotype, MAP4, and Stathmin primer
and probe sequences used in real-time PCR experiments.

| Beta-tubulin | | | |
|---|---|---|---|
| isotype gene | Forward Primer | Probe | Reverse Primer |
| Class I beta-tubulin | ACCTCAGGCTTCTCAGTTCCC | TAGCCGTCTTACTCAACTGCCCCTTTCC | CAGCAAACACAAATTCTGAGGG |
| isotype | (SEQ ID NO: 15) | (SEQ ID NO: 16) | (SEQ ID NO: 17) |
| Class II beta-tubulin | GTGGAAGGAAAGAAGCATGGTC | ACTTTAGGTGTGCGCTGGGTCTCTGG | GTGACAGGCAACAGTGAAGAGC |
| isotype | (SEQ ID NO: 18) | (SEQ ID NO: 19) | (SEQ ID NO: 20) |
| Class III beta-tubulin | CCTCGTCCTCCCCACCTAG | CCACGTGTGAGCTGCTCCTGTCTCTG | AGGCCTGGAGCTGCAATAAG |
| isotype | (SEQ ID NO: 21) | (SEQ ID NO: 22) | (SEQ ID NO: 23) |
| Class IVa beta-tubulin | TCTGACCTTTGATCCGCTAGG | CCCCCATCTCTGAACCCTAGAGCCC | TCAGCCTTGGAGGGAAAGC |
| isotype | (SEQ ID NO: 24) | (SEQ ID NO: 25) | (SEQ ID NO: 26) |
| Class IVb beta-tubulin | GGAAGCAGTGTGAACTCTTTATTCAC | CCCAGCCTGTCCTGTGGCCTG | CAGCAAGTGCACACAGTGGG |
| isotype | (SEQ ID NO: 27) | (SEQ ID NO: 28) | (SEQ ID NO: 29) |
| Class V beta-tubulin | CCCTGGTGCCTCCTACCCT | TGGCCCTGAATGGTGCACTGGTTT | GGGCCGACACCAACACAA |
| isotype | (SEQ ID NO: 30) | (SEQ ID NO: 31) | (SEQ ID NO: 32) |
| Class VI beta-tubulin | TGCACTCACCATTAGCTTCGA | ACAGGGACTGAGGGAGACAGGTGGG | CCCTAATGCCTGTCAGCTGC |
| isotype | (SEQ ID NO: 33) | (SEQ ID NO: 34) | (SEQ ID NO: 35) |
| MAP4 | TGAGCCGGTCAGGCACA | ACCAACCAGTCCACGCTCCAAGGG | GCATACACACAACAAAATGGCA |
| | (SEQ ID NO: 36) | (SEQ ID NO: 37) | (SEQ ID NO: 38) |
| Stathmin | CACAAATGACCGCACGTTCT | TGCCCCGTTTCTTGCCCCAG | GGAAGGAGACAATGCAAACCA |
| | (SEQ ID NO: 39) | (SEQ ID NO: 40) | (SEQ ID NO: 41) |

TABLE 2

Alpha-tubulin isotype and Tau primer and probe sequences
from Applied Biosystems used in real-time PCR experiments.

| | | | | |
|---|---|---|---|---|
| Gene | Forward Primer | Probe | Reverse Primer | Assay ID |

| | | | | |
|---|---|---|---|---|
| TUBA1 | TGCCAACAACTATGCCCGT | TATACCATTGGCAAGGAGATCATTGACCCAGT | TGGAACACCAGGAAGCCCT | Hs00428633_ml |
| | (SEQ ID NO: 42) | (SEQ ID NO: 43) | (SEQ ID NO: 44) | |
| TUBA2 | TGGTGCCCAAGGATGTGAA | CTATTGCTGCCATCAAGACCAAGAGGACC | GTTGATGCCCACCTTGAAGC | Hs00606400_ml |
| | (SEQ ID NO: 45) | (SEQ ID NO: 46) | (SEQ ID NO: 47) | |
| TUBA3 | CCTCGTGTTGGACCGAATTC | TGGCCGACCAGTGCACGGG | TGGAAAACCAAGAAGCCCTG | Hs00362387_ml |
| | (SEQ ID NO: 48) | (SEQ ID NO: 49) | (SEQ ID NO: 50) | |
| TUBA4 | GGAAGGAGTTCATCGACCTGC | ACCGGATTCGGAAGCTGGCTGAC | TGGAACACCAGGAAGCCCT | Hs0O257705_ml |
| | (SEQ ID NO: 51) | (SEQ ID NO: 52) | (SEQ ID NO: 53) | |
| TUBA6 | CCCGAGGGCACTACACCAT | CAGTGCACCGGTCTTCAGGGCTTC | AACCAGTTCCCCCACCAAAG | Hs00733770_ml |
| | (SEQ ID NO: 54) | (SEQ ID NO: 55) | (SEQ ID NO: 56) | |
| TUBA8 | TGGTGCCCAAGGATGTGAA | TTGCTGCCATCAAGACCAAGAGGACC | GTTGATGCCCACCTTGAAGC | Hs00251803_ml |
| | (SEQ ID NO: 57) | (SEQ ID NO: 58) | (SEQ ID NO: 59) | |
| Tau | | | | Hs00213491_ml |

TABLE 3
Sensitivity of human breast cancer cell lines to tubulin-
binding agents in cell growth inhibition assay.

| | Average IC₅₀ (nM) | | | |
|---|---|---|---|---|
| Cell line | E7389 | E7974 | Paclitaxel | Vinblastine |
| AU565 | 0.65 | 1.84 | 2.50 | 1.05 |
| BT-20 | 0.82 | 1.99 | 2.63 | 1.15 |
| MCF-7 | 1.45 | 2.17 | 2.31 | 0.89 |
| MDA-MB-231 | 1.75 | 2.69 | 5.07 | 1.60 |
| MDA-MB-435 | 0.29 | 0.66 | 2.42 | 0.43 |
| MDA-MB-468 | 0.45 | 1.00 | 4.30 | 0.82 |
| HCC38 | 0.40 | 1.09 | 2.94 | 0.73 |
| HCC70 | 0.63 | 1.41 | 2.25 | 0.80 |
| HCC1143 | 0.43 | 1.10 | 2.11 | 0.53 |
| HCC1419 | 1.59 | 3.85 | 5.41 | 1.70 |
| HCC1428 | 0.53 | 1.21 | 5.03 | 0.55 |
| HCC1500 | 0.56 | 0.63 | 2.48 | 0.56 |
| HCC1806 | 0.74 | 2.19 | 2.10 | 0.86 |
| HCC1954 | 0.37 | 1.59 | 8.48 | 0.70 |
| HC-2218 | 1.14 | 3.66 | 6.72 | 1.56 |
| UACC-812 | 1.17 | 2.83 | 1.46 | 1.11 |
| UACC-893 | 0.31 | 2.04 | 2.73 | 1.32 |
| ZR-75-1 | 1.19 | 2.27 | 3.35 | 1.72 |
| ZR-75-30 | 1.80 | 4.27 | 4.06 | 2.40 |

TABLE 4
Effect of verapamil on potency of paclitaxel as evidence
of no P-glycoprotein expression in the cell lines

| | | Paclitaxel IC₅₀ |
|---|---|---|
| | Paclitaxel IC₅₀ | (+10 μM |
| Cell Line | (no verapamil) | verapamil) |
| ZR-75-30 | 3.9 | 4.1 |
| HCC-2218 | 5.6 | 5.0 |
| HCC-1419 | 5.2 | 7.0 |
| AU-565 | 5.4 | 4.8 |
| BT-20 | 3.9 | 2.7 |
| HCC-1428 | 9.0 | 9.5 |
| HCC-1806 | 1.8 | 1.9 |
| HCC-1954 | 11.7 | 11.7 |
| UACC-812 | 2.7 | 1.9 |
| MDA-MB-435 | 2.6 | 2.7 |
| ZR-75-1 | 5.3 | 5.9 |
| MDA-MB-231 | 4.9 | 5.1 |
| MDA-MB-468 | 5.1 | 5.4 |
| HCC-70 | 2.0 | 1.9 |
| HCC-1143 | 2.8 | 2.9 |
| UACC-893 | 6.3 | 4.1 |
| MCF-7 | 3.3 | 2.7 |
| HCC-1500 | 4.6 | 3.2 |
| HCC-38 | 5.9 | 6.2 |

TABLE 5
Expression level of beta-tubulin isotypes, stathmin and MAP4 genes in human breast cancer cell lines.

| | Beta-tubulin isotypes | | | | | | | | |
|---|---|---|---|---|---|---|---|---|---|
| Cell Line | I | II | III | IVa | IVb | V | VI | stathmin | MAP4 |
| AU565* | 1.00E+00 | 1.00E+00 | 1.00E+00 | 1.00E+00 | 1.00E+00 | 1.00E+00 | 1.00E+00 | 1.00E+00 | 1.00E+00 |
| BT-20 | 1.42E+00 | 1.28E−01 | 2.93E+00 | 4.07E+00 | 4.34E+00 | 1.81E+00 | 2.04E+00 | 2.93E+00 | 1.73E+00 |
| MCF-7 | 1.72E+00 | 2.64E+01 | 8.21E+00 | 1.64E+01 | 3.51E+00 | 4.32E+00 | 9.87E−01 | 2.77E+00 | 2.55E+00 |
| MDA-MB-231 | 4.89E+00 | 1.79E+02 | 2.18E+01 | 7.56E+00 | 5.18E+00 | 2.88E+01 | 4.38E−01 | 1.43E+01 | 4.86E+00 |
| MDA-MB-435 | 2.55E+00 | 4.83E+01 | 1.52E+01 | 1.05E+04 | 2.54E+00 | 1.16E+00 | 9.83E−01 | 4.20E+00 | 4.59E+00 |
| MDA-MB-468 | 6.55E+00 | 3.87E+02 | 2.79E+01 | 1.40E+00 | 2.56E+00 | 1.05E+01 | 4.86E−01 | 1.35E+01 | 2.08E+00 |
| HCC-38 | 3.37E+00 | 4.41E+04 | 1.28E+02 | 1.22E+02 | 4.51E+00 | 4.99E+01 | 1.55E+00 | 1.41E+01 | 5.10E+00 |
| HCC-70 | 3.10E+00 | 6.15E+00 | 4.18E+01 | 4.23E−01 | 3.46E+00 | 9.21E+00 | 1.02E+00 | 1.47E+01 | 5.31E+00 |
| HCC-1143 | 3.85E+00 | 1.97E+03 | 5.73E+01 | 5.29E+02 | 2.92E+00 | 1.72E+01 | 1.17E+00 | 7.73E+00 | 4.53E+00 |
| HCC-1419 | 7.91E−01 | 8.78E+00 | 4.01E−01 | 1.59E+01 | 2.85E+00 | 3.79E−01 | 7.41E+00 | 1.00E+00 | 1.29E+00 |
| HCC-1428 | 1.29E+00 | 1.71E+01 | 5.65E+00 | 1.41E+01 | 2.62E+00 | 3.04E+00 | 2.17E+00 | 2.93E+00 | 9.73E−01 |
| HCC-1500 | 2.68E+00 | 2.30E+03 | 7.72E+01 | 5.92E−01 | 6.23E+00 | 5.25E−01 | 3.91E+00 | 5.62E+00 | 2.87E+00 |
| HCC-1806 | 1.61E+00 | 5.13E+02 | 1.57E+01 | 6.70E+01 | 2.11E+00 | 7.54E+00 | 3.56E−01 | 2.00E+00 | 1.54E+00 |
| HCC-1954 | 1.25E+00 | 7.31E+01 | 1.09E+01 | 1.12E+01 | 2.22E+00 | 2.59E+00 | 1.73E+00 | 9.33E−01 | 3.16E+00 |
| HCC-2218 | 1.56E+00 | 6.58E−01 | 3.21E−01 | 7.43E+01 | 2.93E+00 | 2.01E−02 | 1.67E+00 | 5.82E−01 | 1.16E+00 |
| UACC-812 | 9.73E−01 | 8.80E+01 | 1.27E+01 | 4.24E+00 | 2.38E+00 | 3.31E+00 | 8.71E+00 | 6.51E−01 | 1.62E+00 |
| UACC-893 | 1.89E+00 | 3.72E+00 | 6.32E+01 | 4.91E+01 | 3.17E+00 | 2.02E+00 | 5.96E+00 | 8.53E−01 | 4.54E−01 |
| ZR-75-1 | 7.53E−01 | 1.60E+02 | 1.55E+01 | 8.14E+01 | 2.66E+00 | 4.88E+00 | 1.41E+00 | 1.96E+00 | 6.42E−01 |
| ZR-75-30 | 1.43E+00 | 2.38E+00 | 1.73E−01 | 1.03E+00 | 3.04E+00 | 1.74E+00 | 2.95E+00 | 8.59E−01 | 2.36E+00 |

Alpha-tubulin isotypes

| Cell Line | TUBA1 | TUBA3 | TUBA6 | TUBA8 | Tau |
|---|---|---|---|---|---|
| AU565* | 1.00E+00 | 1.00E+00 | 1.00E+00 | 1.00E+00 | 1.00E+00 |
| BT-20 | 4.87E+00 | 4.09E+01 | 2.41E+00 | 2.79E−01 | 6.27E−01 |
| MCF-7 | 2.52E−02 | 1.05E+04 | 3.39E+00 | 6.53E−01 | 8.31E+01 |
| MDA-MB-231 | 7.07E−01 | 6.86E+03 | 5.43E+00 | 9.54E−02 | 1.18E+00 |
| MDA-MB-435 | 2.08E+00 | 1.15E+04 | 2.41E+00 | 4.68E−03 | 3.07E+01 |
| MDA-MB-468 | 2.28E−01 | 2.04E+03 | 3.75E+00 | 3.13E+01 | 3.84E+00 |
| HCC-38 | 1.17E+01 | 5.63E+04 | 5.03E+00 | 2.77E+01 | 8.26E+00 |
| HCC-70 | 7.41E+00 | 5.44E+03 | 3.35E+00 | 1.11E+00 | 3.03E−01 |
| HCC-1143 | 1.04E+01 | 1.41E+04 | 2.70E+00 | 4.33E+00 | 1.92E+00 |
| HCC-1419 | 1.30E+00 | 6.20E+01 | 1.64E+00 | 1.42E+00 | 9.61E−02 |
| HCC-1428 | 1.29E+00 | 1.14E+04 | 1.69E+00 | 2.21E+00 | 2.25E+02 |
| HCC-1500 | 1.31E+00 | 2.81E+04 | 3.77E+00 | 1.06E+00 | 6.42E+02 |
| HCC-1806 | 4.45E+00 | 1.49E+02 | 3.39E+00 | 1.71E+00 | 2.11E+00 |
| HCC-1954 | 2.58E+00 | 2.14E+02 | 2.10E+00 | 1.65E−01 | 3.90E+00 |
| HCC-2218 | 7.45E−01 | 4.00E+02 | 2.40E+00 | 1.87E+00 | 5.25E+01 |
| UACC-812 | 1.52E−01 | 1.92E+03 | 1.02E+00 | 1.43E+00 | 3.20E+01 |
| UACC-893 | 2.95E+00 | 1.42E+01 | 2.91E+00 | 7.29E+00 | 3.22E+01 |
| ZR-75-1 | 1.02E+00 | 7.18E+03 | 1.60E+00 | 4.74E+00 | 4.64E+01 |
| ZR-75-30 | 5.17E+00 | 2.11E+03 | 2.59E+00 | 5.93E−01 | 9.43E+00 |

*Expression level of gene of interest in AU565 cell line was chosen as a reference (baseline) control for calculations.

TABLE 6
Correlation analysis of sensitivity of human breast cancer cell lines
to tubulin-binding agents with expression level of beta-tubulin isotypes,
alpha-tubulin isotypes, stathmin, MAP4, and Tau genes.

| | 7389 | | 7974 | | paclitaxel | | vinblastine | |
|---|---|---|---|---|---|---|---|---|
| Gene* | r | p | r | p | r | p | r | p |
| Class I | −0.21 | 0.39 | −0.42 | 0.07 | −0.05 | 0.84 | −0.27 | 0.26 |
| Class II | −0.24 | 0.32 | −0.24 | 0.32 | −0.10 | 0.67 | −0.19 | 0.44 |
| Class III | −0.48 | 0.04 | −0.52 | 0.02 | −0.31 | 0.20 | −0.38 | 0.11 |
| Class IVA | −0.28 | 0.24 | −0.32 | 0.18 | −0.17 | 0.50 | −0.31 | 0.19 |
| Class IVB | 0.13 | 0.59 | −0.16 | 0.51 | −0.10 | 0.69 | −0.02 | 0.93 |
| Class V | −0.08 | 0.75 | −0.24 | 0.33 | −0.08 | 0.73 | −0.14 | 0.56 |
| Class VI | 0.20 | 0.42 | 0.38 | 0.11 | −0.08 | 0.74 | 0.26 | 0.28 |
| stathmin | −0.17 | 0.49 | −0.44 | 0.06 | −0.12 | 0.61 | −0.28 | 0.24 |
| MAP4 | −0.15 | 0.54 | −0.40 | 0.09 | −0.11 | 0.66 | −0.37 | 0.12 |
| Tau | −0.15 | 0.53 | −0.34 | 0.15 | −0.09 | 0.7 | −0.30 | 0.22 |
| TUBA1 | −0.33 | 0.17 | −0.24 | 0.31 | −0.26 | 0.27 | −0.20 | 0.41 |
| TUBA3 | −0.28 | 0.24 | −0.46 | 0.05 | −0.21 | 0.37 | −0.38 | 0.11 |
| TUBA6 | −0.00 | 0.99 | −0.24 | 0.31 | −0.05 | 0.85 | −0.12 | 0.62 |
| TUBA8 | −0.35 | 0.14 | −0.33 | 0.17 | −0.03 | 0.91 | −0.18 | 0.46 |

*Class I-VI are beta-tubulin isotype genes
**r is a correlation coefficient. p < 0.05 is a conventional threshold of significance.

TABLE 7
Multiple stepwise linear regression analysis of associations of gene expressions
and sensitivity to tubulin-binding agents.

| | E7389 | | E7974 | | Paclitaxel | | Vinblastine | |
|---|---|---|---|---|---|---|---|---|
| | Forward | Backward | Forward | Backward | Forward | Backward | Forward | Backward |
| | Partial β | Partial β | Partial β | Partial β | Partial β | Partial β | Partial β | Partial β |
| Gene | coefficient | coefficient | coefficient | coefficient | coefficient | coefficient | coefficient | coefficient |
| Overall | 0.87 | 0.76 | 0.93 | 0.87 | 0.19 | 0.00 | 0.79 | 0.70 |
| R²* | | | | | | | | |
| Stathmin | | | −1.24** | −1.07** | | | | |
| MAP4 | | | | −0.46* | | | −0.62* | −0.47* |
| TUBA1 | −0.42* | −0.37** | | | | | | |
| TUBA3 | | | | 0.74* | | | | |
| TUBA6 | | | | | | | | |
| TUBA8 | | | | | | | | |
| Class I | | | | | | | | |
| Class II | | | | | | | | |
| Class III | −0.72* | −1.00*** | −0.53* | −0.48* | | | −0.73* | −0.95*** |
| Class IVa | | | | | | | | |
| Class IVb | −0.68* | 0.48** | 0.51* | 0.33* | | | 0.49* | 0.46** |
| Class V | | 0.49** | | | | | | 0.50* |
| Class VI | | | | | | | | |
| TAU | | | −0.68* | −0.76** | | | | |

Each model is based on the standardized data, and the log₁₀ transformed expression levels of the isotypes.
R² - The proportion of variation in data explained by the regression model.
Class I-VI are beta-tubulin isotype genes.
*significant at P < .05,
**significant at P < .01,
***significant at P < .001.

### Example 2

### Use of Gene Chips in Identifying Patients for Treatment

The present Example describes the use of gene chip microarrays in selecting a patient for treatment using a particular anti-microtubule agent.

A sample from the cancer of the patient is obtained. The mRNA from the cells of the cancer cells is isolated. The isolated mRNA is reverse transcribed to yield cDNA which is then labeled with a fluorescent marker. The labeled cDNA is then incubated with a microarray containing nucleotides specific for tubulin isotypes as well as Tau, MAP4, and stathmin. The arrays is washed repeatedly using increasingly stringent washes to remove unhybridized cDNA. The array is then spun dry. The array with the hybridized labeled cDNA is then analyzes using a laser-scanning microscope. The net signal for each spot was determined by subtracting the local background from the average spot intensity. The signal intensities for each spot are then normalized.

Based on the expression levels seen in the cancer cells, the patient is either selected or not selected for treatment. For example, expression level of the class III isotype of β-tubulin has been shown to correlated with a high sensitivity to E7389 and E7974. Therefore, patients whose cancer cells express elevated levels of the class III isotype of β-tubulin would be suitable candidates for treatment with E7389, E7974, or other analogs of these compounds. In certain instances, the expression level of a particular β-tubulin isotype may rule out a patient for treatment with a particular compound. For example, the expression of the class II isotype of β-tubulin in the patient's cancer may rule the patient for treatment with taxol.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of identifying a patient with cancer for treatment with a chemical compound, the method comprising steps of:
(a) obtaining a sample from the cancer of a patient; and
(b) analyzing the sample for expression levels or protein levels of at least one marker selected from the group consisting of α-tubulin isotypes, β-tubulin isotypes, and microtubule-associated biomolecules, wherein a correlation exists between sensitivity to a chemical compound and expression levels or protein levels of the marker, and
wherein the chemical compound is of the formula (I): 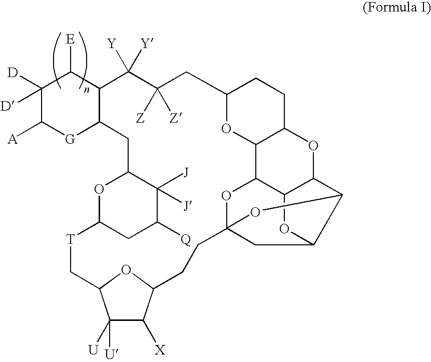
wherein A is a C₁₋₆ saturated or C₂₋₆ unsaturated hydrocarbon skeleton, the skeleton being unsubstituted or having between 1 and 13 substituents selected from the group consisting of cyano, halo, azido, Q₁, and oxo, wherein each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁ and O(CO)NR₂R₁;
wherein each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₄ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl;
wherein each of D and D′ is independently selected from R₃ and OR₃, wherein R₃ is H, C₁₋₃ alkyl, or C₁₋₃ haloalkyl;
wherein the value for n is 1 or 0, thereby forming either a six-membered or five-membered ring, wherein the ring can be unsubstituted or substituted, where E is —R₅ or —OR₅, and can be a heterocyclic radical or a cycloalkyl, wherein G is S, SH₂, NR₆, or preferably O;
wherein each of J and J′ is independently H, C₁₋₆ alkoxy, or C₁₋₆ alkyl; or J and J′ taken together are ═CH₂ or —O-(straight or branched C₁₋₅alkylene or alkylidene)-O—;
wherein Q is C₁₋₃ alkyl;
wherein T is methylene, ethylene, or ethenylene, optionally substituted with (CO)OR₇, where R₇ is H or C₁₋₆ alkyl;
wherein each of U and U′ is independently H, C₁₋₆ alkoxy, or C₁₋₆ alkyl; or U and U′ taken together are ═CH₂ or —O-(straight or branched C₁₋₅alkylene or alkylidene)-O—;
wherein X is H or C₁₋₆ alkoxy;
wherein each of Y and Y′ is independently H or C₁₋₆ alkoxy; or Y and Y′ taken together are ═O, ═CH₂, or —O-(straight or branched C₁₋₅alkylene or alkylidene)-O—;
wherein each of Z and Z′ is independently H or C₁₋₆ alkoxy; or Z and Z′ taken together are ═O, ═CH₂, or —O-(straight or branched C₁₋₅alkylene or alkylidene)-O—; or a pharmaceutically acceptable salt thereof; and
(c) identifying the patient based on expression levels or protein levels of the said at least one marker.

2. The method of claim 1, wherein the chemical compound is of the formula (II):

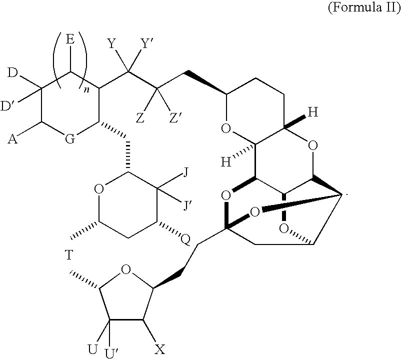

3. The method of claim 1, wherein the chemical compound is of the formula (III):

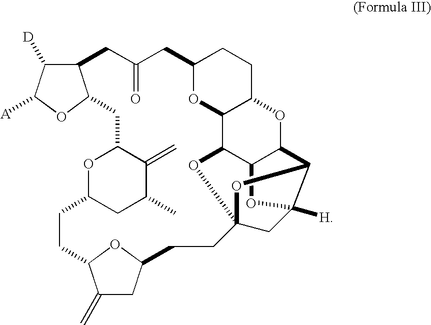

4. The method of claim 1, wherein the chemical compound is of the formula (IV):

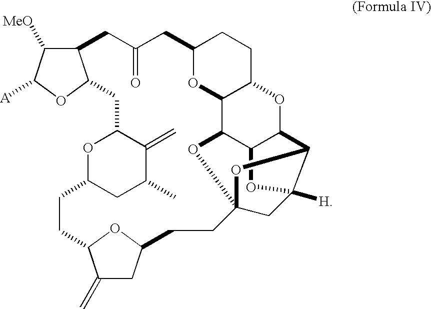

5. The method of claim 1, wherein the chemical compound is of the formula (IV): 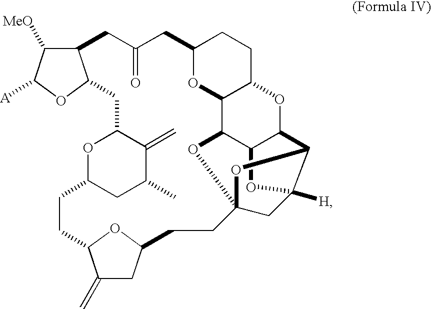
wherein A is a C₁₋₆ saturated or C₂₋₆ unsaturated hydrocarbon skeleton, the skeleton being unsubstituted or having between 1 and 4 substituents selected from the group consisting of azido, hydroxy, OR₁, NH₂, NR₁R₂, NR₂(CO)R₁, NR₂(CO)(CO)R₁, NR₄(CO)NR₂R₁, and NR₂(CO)OR₁;
wherein each of R₁, R₂, and R₄ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₄ alkoxy-C₆ aryl, C₆₋₁₀ aryl-C₁₋₆ alkyl, C₁₋₆ alkyl-C₆₋₁₀aryl, C₆₋₁₀ haloaryl-C₁₋₆ alkyl, C₁₋₆ alkyl-C₆₋₁₀ haloaryl, (C₁₋₃ alkoxy-C₆aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl.

6. The method of claim 1, wherein the chemical compound is of the formula (V):

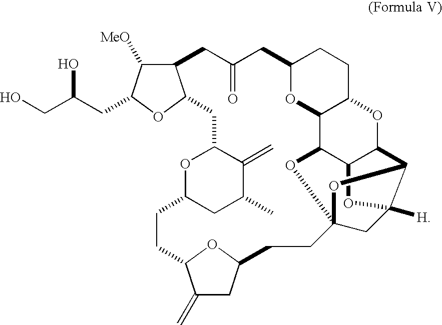

7. The method of claim 1, wherein the chemical compound is of the formula (VI):

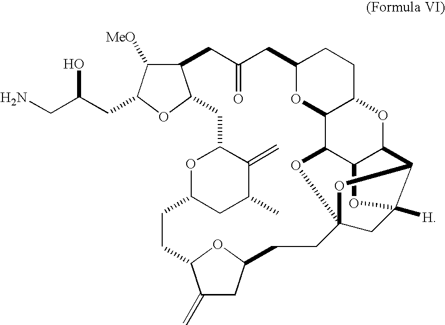

8. The method of claim 1, wherein the marker is selected from the group consisting of α-tubulin isotypes.

9. The method of claim 1, wherein the marker is selected from the group consisting of β-tubulin isotypes.

10. The method of claim 1, wherein the marker is selected from the group consisting of class 1 α-tubulin isotype (TUBA3/b-α1), class 6 α-tubulin isotype (TUBA6), class III β-tubulin isotype (Hβ4/TUBB4), class IVa β-tubulin isotype (Hβ5/TUBB5), class IVb β-tubulin isotype (Hβ2), class V β-tubulin isotype (5-beta/Beta V), and class VI β-tubulin isotype (Hβ1/TUBB1).

11. The method of claim 1, wherein the marker is selected from the group consisting of class III β-tubulin isotype (Hβ4/TUBB4), class IVb β-tubulin isotype (Hβ2), class V β-tubulin isotype (5-beta/Beta V), and class VI β-tubulin isotype (Hβ1/TUBB1).

12. The method of claim 1, wherein the marker is class III β-tubulin isotype (Hb4/TUBB4), stathmin, or MAP4.

13-14. (canceled)

15. The method of claim 1, wherein the expression levels or protein levels of at least two or three markers are analyzed.

16. The method of claim 1, wherein the expression levels or protein levels of at least two or three markers are analyzed, said at least two markers being selected from the group consisting of class 1 α-tubulin isotype (TUBA3/b-α1), class 6 α-tubulin isotype (TUBA6), class III β-tubulin isotype (Hβ4/TUBB4), class IVa β-tubulin isotype (Hβ5/TUBB5), class IVb β-tubulin isotype (Hβ2), class V β-tubulin isotype (5-beta/Beta V), class VI β-tubulin isotype (Hβ1/TUBB1), stathmin, and MAP4.

17-18. (canceled)

19. The method of claim 1, wherein the cancer is selected from the group consisting of breast cancer, ovarian cancer, and lung cancer.

20. (canceled)

21. The method of claim 1, wherein the cancer is a multi-drug resistant cancer, the cells of the cancer express P-glycoprotein (Pgp), or the cancer is paclitaxel-resistant cancer.

22-23. (canceled)

24. The method of claim 1, wherein the step of obtaining a sample from the cancer comprises obtaining a biopsy sample of the cancer, a sample of RNA from the cancer, a sample of protein from the cancer, or a sample of cells from the cancer.

25. (canceled)

26. The method of claim 24, wherein the step of obtaining a sample from the cancer comprises obtaining a sample of RNA from the cancer, and the method further comprises reverse transcribing the RNA into cDNA after obtaining the sample of RNA.

27. The method of 26, further comprising steps of
performing PCR on the cDNA using primers specific for the marker; and
determining the expression of the marker.

28. The method of claim 24, wherein the step of obtaining a sample from the cancer comprises obtaining a sample of RNA from the cancer, and the method further comprises steps of
contacting the cDNA with an array of probes specific for the markers selected from the group consisting of α-tubulin isotypes, β-tubulin isotypes, and microtubule-associated proteins; and
quantifying the expression levels of the markers.

29. (canceled)

30. The method of claim 24, wherein the step of obtaining a sample from the cancer comprises obtaining a sample of protein from the cancer, and the method further comprises the step of:
contacting the sample with antibodies specific for the marker or analyzing the sample for the marker using mass spectroscopy.

31-32. (canceled)

33. The method of claim 1, wherein the step of identifying the patient based on expression levels or protein levels of the said at least one marker comprises identifying the patient based on increased levels of the said at least one marker.

34. The method of claim 33, wherein the increased level of at least one marker is at least twice, three times, or five times the level in control cells.

35-36. (canceled)

37. A method of selecting a compound for treating a patient with cancer based on the expression level or protein level of at least one marker selected from the group consisting of α-tubulin isotypes, β-tubulin isotypes, and microtubule-associated biomolecules, the method comprising steps of:
administering to the patient a compound of the formula (I): 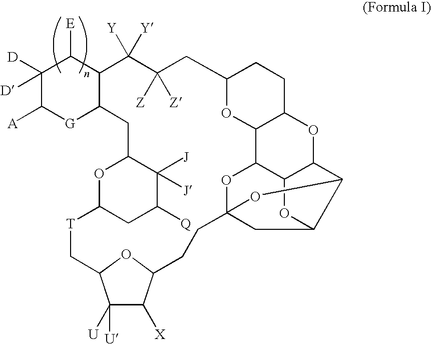
wherein A is a C₁₋₆ saturated or C₂₋₆ unsaturated hydrocarbon skeleton, the skeleton being unsubstituted or having between 1 and 13 substituents selected from the group consisting of cyano, halo, azido, Q₁, and oxo, wherein each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁ and O(CO)NR₂R₁;
wherein each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₄ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl;
wherein each of D and D′ is independently selected from R₃ and OR₃, wherein R₃ is H, C₁₋₃ alkyl, or C₁₋₃ haloalkyl;
wherein the value for n is 1 or 0, thereby forming either a six-membered or five-membered ring, wherein the ring can be unsubstituted or substituted, where E is —R₅ or —OR₅, and can be a heterocyclic radical or a cycloalkyl, wherein G is S, SH₂, NR₆, or preferably O;
wherein each of J and J′ is independently H, C₁₋₆alkoxy, or C₁₋₆ alkyl; or J and J′ taken together are ═CH₂ or —O-(straight or branched C₁₋₅alkylene or alkylidene)-O—;
wherein Q is C₁₋₃ alkyl;
wherein T is methylene, ethylene, or ethenylene, optionally substituted with (CO)OR₇, where R₇ is H or C₁₋₆ alkyl;
wherein each of U and U′ is independently H, C₁₋₆ alkoxy, or C₁₋₆ alkyl; or U and U′ taken together are ═CH₂ or —O-(straight or branched C₁₋₅alkylene or alkylidene)-O—;
wherein X is H or C₁₋₆ alkoxy;
wherein each of Y and Y′ is independently H or C₁₋₆ alkoxy; or Y and Y′ taken together are ═O, ═CH₂, or —O-(straight or branched C₁₋₅alkylene or alkylidene)-O—;
wherein each of Z and Z′ is independently H or C₁₋₆ alkoxy; or Z and Z′ taken together are ═O, ═CH₂, or —O-(straight or branched C₁₋₅alkylene or alkylidene)-O—; or a pharmaceutically acceptable salt thereof;
based on the expression level or protein level of at least one marker selected from the group consisting of α-tubulin isotypes, β-tubulin isotypes, and microtubule-associated biomolecules.

38. The method of claim 37, wherein the chemical compound is of the formula (II):

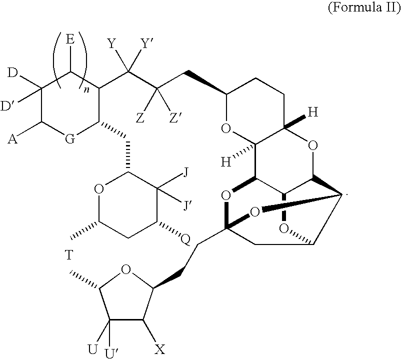

39. The method of claim 37, wherein the chemical compound is of the formula (III):

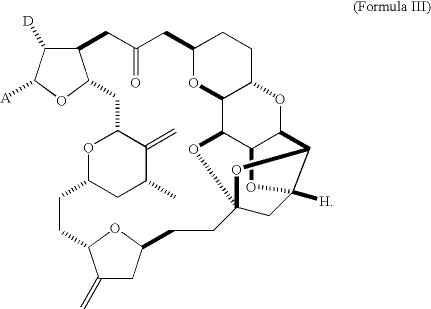

40. The method of claim 37, wherein the chemical compound is of the formula (IV):

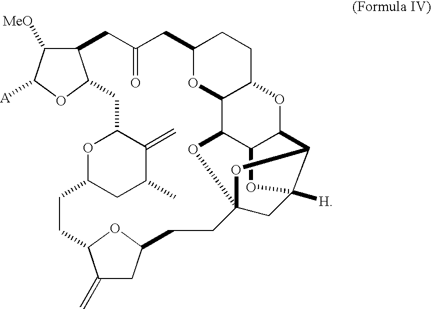

41. The method of claim 37, wherein the chemical compound is of the formula (IV): 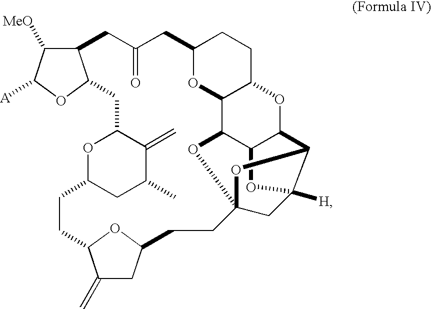
wherein A is a C₁₋₆ saturated or C₂₋₆ unsaturated hydrocarbon skeleton, the skeleton being unsubstituted or having between 1 and 4 substituents selected from the group consisting of azido, hydroxy, OR₁, NH₂, NR₁R₂, NR₂(CO)R₁, NR₂(CO)(CO)R₁, NR₄(CO)NR₂R₁, and NR₂(CO)OR₁;
wherein each of R₁, R₂, and R₄ is independently selected from H, C₁₋₆ alkyl, C₁₋₆ haloalkyl, C₁₋₆ hydroxyalkyl, C₁₋₆ aminoalkyl, C₆₋₁₀ aryl, C₆₋₁₀ haloaryl, C₆₋₁₀ hydroxyaryl, C₁₋₄ alkoxy-C₆ aryl, C₆₋₁₀ aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl.

42. The method of claim 37, wherein the chemical compound is of the formula (V):

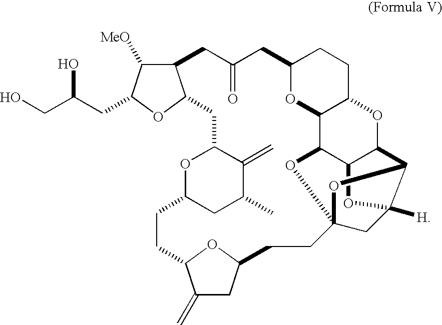

43. The method of claim 37, wherein the chemical compound is of the formula (VI):

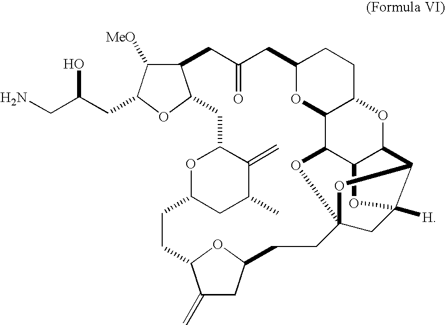

44. The method of claim 37, wherein the marker is selected from the group consisting of α-tubulin isotypes.

45. The method of claim 37, wherein the marker is selected from the group consisting of β-tubulin isotypes.

46. The method of claim 37, wherein the marker is selected from the group consisting of class 1 α-tubulin isotype (TUBA3/b-α1), class 6 α-tubulin isotype (TUBA6), class III β-tubulin isotype (Hβ4/TUBB4), class IVa β-tubulin isotype (Hβ5/TUBB5), class IVb β-tubulin isotype (Hβ2), class V β-tubulin isotype (5-beta/Beta V), and class VI β-tubulin isotype (Hβ1/TUBB1).

47. The method of claim 37, wherein the marker is selected from the group consisting of class III β-tubulin isotype (Hβ4/TUBB4), class IVb β-tubulin isotype (Hβ2), class V β-tubulin isotype (5-beta/Beta V), and class VI β-tubulin isotype (Hβ1/TUBB1).

48. The method of claim 37, wherein the marker is class III β-tubulin isotype (Hb4/TUBB4), stathmin, or MAP4.

49-50. (canceled)

51. A polynucleotide selected from the group consisting of the following sequences:

| | |
|---|---|
| ACCTCAGGCTTCTCAGTTCCC; | (SEQ ID NO: 15) |
| TAGCCGTCTTACTCAACTGCCCCTTTCC; | (SEQ ID NO: 16) |
| CAGCAAACACAAATTCTGAGGG; | (SEQ ID NO: 17) |
| GTGGAAGGAAAGAAGCATGGTC ; | (SEQ ID NO: 18) |
| ACTTTAGGTGTGCGCTGGGTCTCTGG; | (SEQ ID NO: 19) |
| GTGACAGGCAACAGTGAAGAGC; | (SEQ ID NO: 20) |
| CCTCGTCCTCCCCACCTAG; | (SEQ ID NO: 21) |
| CCACGTGTGAGCTGCTCCTGTCTCTG; | (SEQ ID NO: 22) |
| AGGCCTGGAGCTGCAATAAG; | (SEQ ID NO: 23) |
| TCTGACCTTTGATCCGCTAGG; | (SEQ ID NO: 24) |
| CCCCCATCTCTGAACCCTAGAGCCC; | (SEQ ID NO: 25) |
| TCAGCCTTGGAGGGAAAGC; | (SEQ ID NO: 26) |
| GGAAGCAGTGTGAACTCTTTATTCAC; | (SEQ ID NO: 27) |
| CCCAGCCTGTCCTGTGGCCTG; | (SEQ ID NO: 28) |
| CAGCAAGTGCACACAGTGGG; | (SEQ ID NO: 29) |
| CCCTGGTGCCTCCTACCCT; | (SEQ ID NO: 30) |
| TGGCCCTGAATGGTGCACTGGTTT; | (SEQ ID NO: 31) |
| GGGCCGACACCAACACAA; | (SEQ ID NO: 32) |
| TGCACTCACCATTAGCTTCGA; | (SEQ ID NO: 33) |
| ACAGGGACTGAGGGAGACAGGTGGG; | (SEQ ID NO: 34) |
| and | |
| CCCTAATGCCTGTCAGCTGC. | (SEQ ID NO: 35) |

52. A method of establishing a correlation between expression of a marker gene and susceptibility to a chemical compound, the method comprising steps of:
providing a cell;
contacting the cell with a compound of the formula (I): 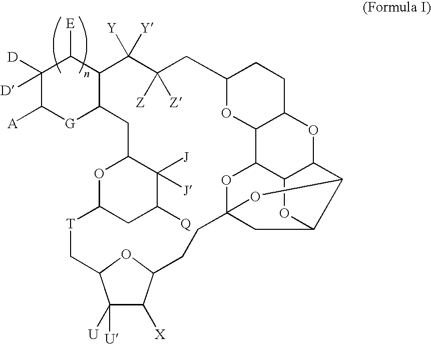
wherein A is a C₁₋₆ saturated or C₂₋₆ unsaturated hydrocarbon skeleton, the skeleton being unsubstituted or having between 1 and 13 substituents selected from the group consisting of cyano, halo, azido, Q₁, and oxo, wherein each Q₁ is independently selected from OR₁, SR₁, SO₂R₁, OSO₂R₁, NR₂R₁, NR₂(CO)R₁, NR₂(CO)(CO)R₁, NR₄(CO)NR₂R₁, NR₂(CO)OR₁, (CO)OR₁, O(CO)R₁, (CO)NR₂R₁ and O(CO)NR₂R₁;
wherein each of R₁, R₂, R₄, R₅, and R₆ is independently selected from H, C₁₋₆ alkyl, C₁₋₆ haloalkyl, C₁₋₆ hydroxyalkyl, C₁₋₆ aminoalkyl, C₆₋₁₀aryl, C₆₋₁₀ haloaryl, C₆₋₁₀ hydroxyaryl, C₁₋₄ alkoxy-C₆ aryl, C₆₋₁₀aryl-C₁₋₆ alkyl, C₁₋₆ alkyl-C₆₋₁₀ aryl, C₆₋₁₀ haloaryl-C₁₋₆ alkyl, C₁₋₆ alkyl-C₆₋₁₀ haloaryl, (C₁₋₃ alkoxy-C₆ aryl)-C₁₋₃ alkyl, C₂₋₉heterocyclic radical, C₂₋₉heterocyclic radical-C₁₋₆ alkyl, C₂₋₉heteroaryl, and C₂₋₉heteroaryl-C₁₋₆ alkyl;
wherein each of D and D′ is independently selected from R₃ and OR₃, wherein R₃ is H, C₁₋₃ alkyl, or C₁₋₃ haloalkyl;
wherein the value for n is 1 or 0, thereby forming either a six-membered or five-membered ring, wherein the ring can be unsubstituted or substituted, where E is —R₅ or —OR₅, and can be a heterocyclic radical or a cycloalkyl, wherein G is S, SH₂, NR₆, or preferably O;
wherein each of J and J′ is independently H, C₁₋₆ alkoxy, or C₁₋₆ alkyl; or J and J′ taken together are ═CH₂ or —O-(straight or branched C₁₋₅alkylene or alkylidene)-O—;
wherein Q is C₁₋₃ alkyl;
wherein T is methylene, ethylene, or ethenylene, optionally substituted with (CO)OR₇,
where R₇ is H or C₁₋₆ alkyl;
wherein each of U and U′ is independently H, C₁₋₆ alkoxy, or C₁₋₆ alkyl; or U and U′ taken together are ═CH₂ or —O-(straight or branched C₁₋₅alkylene or alkylidene)-O—;
wherein X is H or C₁₋₆ alkoxy;
wherein each of Y and Y′ is independently H or C₁₋₆ alkoxy; or Y and Y′ taken together are ═O, ═CH₂, or —O-(straight or branched C₁₋₅alkylene or alkylidene)-O—;
wherein each of Z and Z′ is independently H or C₁₋₆ alkoxy; or Z and Z′ taken together are ═O, ═CH₂, or —O-(straight or branched C₁₋₅alkylene or alkylidene)-O—; or a pharmaceutically acceptable salt thereof;
assaying the cell for growth inhibition;
determining the expression of tubulin isotypes or microtubule-associated genes in the cell; and
determining a correlation between expression levels or protein levels of one or more tubulin isotypes or microtubule-associated biomolecules and susceptibility to the compound tested.

53-60. (canceled)
